(19) United States
(12) Patent Application Publication
(10) Pub. No.: US 2022/0233080 A1
(43) Pub. Date: Jul. 28, 2022

(54) SYSTEMS AND METHODS FOR MONITORING ONE OR MORE PHYSIOLOGICAL PARAMETERS USING BIO-IMPEDANCE

(71) Applicant: The Texas A&M University System, College Station, TX (US)

(72) Inventors: Roozbeh Jafari, Austin, TX (US); Kaan Sel, Bryan, TX (US); Bassem Ibrahim, College Station, TX (US)

(73) Assignee: The Texas A&M University System, College Station, TX (US)

(21) Appl. No.: 17/609,489

(22) PCT Filed: May 8, 2020
(86) PCT No.: PCT/US2020/032180

§ 371 Date: Nov. 8, 2021

Related U.S. Application Data
(60) Provisional application No. 62/845,114, filed on May 8, 2019.

(51) Int. Cl. A61B 5/0205; A61B 5/053; A61B 5/00; A61B 5/273

(52) U.S. Cl. CPC A61B 5/0205; A61B 5/053; A61B 2562/04; A61B 5/7235; A61B 5/273; A61B 5/6833

(57) ABSTRACT

A system for monitoring one or more physiological parameters includes a plurality of patches positionable at a plurality of locations on a surface of a body, wherein the plurality of patches are electrically isolated from each other, wherein a first patch of the plurality of patches includes a transmitter configured to inject an electrical signal into the body at a first location on the surface of the body, and wherein a second patch of the plurality of patches includes a sensor configured to detect a bio-impedance signal at a second location on the surface of the body that is spaced from the first location, and wherein the bio-impedance signal is induced within the body by the electrical signal injected from the first patch which is electrically isolated from the second patch.

### CROSS-REFERENCE TO RELATED APPLICATIONS

This application claims benefit of U.S. provisional patent application Ser. No. 62/845,114 filed May 8, 2019, and entitled “Wirelessly Coupled Bio-Impedance Patches,” which is hereby incorporated herein by reference in its entirety.

### STATEMENT REGARDING FEDERALLY SPONSORED RESEARCH OR DEVELOPMENT

Not applicable.

### BACKGROUND

Prevention and treatment of many disorders may require sustainable and long-term health tracking. For instance, in the case of cardiovascular and respiratory diseases, the symptoms of many complications may be observed through anomalies in the hemodynamic (e.g., heart rate (HR), heart rate variability, blood pressure (BP), respiration rate (RR), etc.) and respiratory physiological parameters (e.g., respiration rate, pulmonary volumes) of a patient. Continuous and reliable monitoring of a patient's physiological parameters may allow for the early diagnosis, management, or even prevention of disease.

A variety of techniques may be employed for monitoring one or more physiological parameters of a patient. For example, nocturnal polysomnography (PSG), which monitors chest wall and upper abdominal wall movements through piezoelectric sensors, and capnography, which monitors CO₂ and O₂ gas exchanges using nasal sensors or face masks, are commonly used techniques for respiration monitoring. Additionally, wearable systems may be used for monitoring HR and other hemodynamic parameters through electrocardiogram (ECG) signals, which are obtained electrodes placed on the patient's skin that monitor electrical activity of the heart, or photoplethysmography (PPG) signals, which are obtained through measuring the intensity of reflected and/or transmitted light applied to the patient's skin. Bio-impedance is another technique which may be used to monitor a patient's hemodynamic and/or physiological parameters. Techniques employing bio-impedance (“Bio-Z”) may include stimulating a patient's epidermis with alternating electrical current (AC) and measuring a voltage difference induced by the AC stimulation across two points on the patient's body.

### BRIEF SUMMARY OF THE DISCLOSURE

An embodiment of a system for monitoring one or more physiological parameters comprises a plurality of patches positionable at a plurality of locations on a surface of a body, wherein the plurality of patches are electrically isolated from each other, wherein a first patch of the plurality of patches comprises a transmitter configured to inject an electrical signal into the body at a first location on the surface of the body, and wherein a second patch of the plurality of patches comprises a sensor configured to detect a bio-impedance signal at a second location on the surface of the body that is spaced from the first location, and wherein the bio-impedance signal is induced within the body by the electrical signal injected from the first patch which is electrically isolated from the second patch. In some embodiments, the transmitter of the first patch is configured to inject an alternating current at a first frequency, the sensor of the second patch is configured to detect the bio-impedance signal from a voltage induced by the injected alternating current, and the second patch comprises a transmitter configured to inject an alternating current at a second frequency that is different from the first frequency. In some embodiments, the first patch comprises sensor configured to detect a bio-impedance signal from the body that is induced by the alternating currents injected from the first patch and the second patch. In certain embodiments, the system further comprises a controller coupled to the second patch and configured to estimate the one or more physiological parameters based on the bio-impedance signal. In certain embodiments, the controller is configured to adjust at least one of a frequency, an amplitude, and a shape of the electrical signal injected by the transmitter. In some embodiments, at least one of the one or more physiological parameters estimated by the controller corresponds to at least one of heart activity and respiratory activity of a patient. In some embodiments, the one or more physiological parameters comprises a plurality of physiological parameters, and wherein the controller is configured to apply a source separation algorithm on the bio-impedance signal to separate a first physiological parameter from the plurality of physiological parameters. In certain embodiments, the controller is configured to apply a demodulation algorithm to the bio-impedance signal prior to the application of the source separation algorithm to the bio-impedance signal. In certain embodiments, each of the plurality of patches are independently grounded to the body. In some embodiments, each of the plurality of patches comprises a ground electrode in electrical contact with the surface of the body. In some embodiments, at least one of the plurality of patches comprises a sensors configured to independently detect a bio-impedance signal from the body induced by the electrical signal injected from the second patch. In certain embodiments, the transmitter of the first patch is configured to inject an alternating voltage at a first frequency, the sensor of the second patch is configured to detect the bio-impedance signal from a current induced by the injected alternating voltage, the second patch comprises a transmitter configured to inject an alternating voltage at a second frequency that is different from the first frequency, and the first patch comprises sensor configured to detect a bio-impedance signal from the body that is induced by the alternating voltages injected from the first patch and the second patch.

An embodiment of a system for monitoring one or more physiological parameters comprises a plurality of patches configured to releasably couple with a surface of a body at a plurality of locations, wherein each of the plurality of patches are independently electrically grounded to the body, wherein a first patch of the plurality of patches comprises a transmitter configured to inject an electrical signal into the body at a first location on the surface of the body, and wherein a second patch of the plurality of patches comprises a sensor configured to detect a bio-impedance signal at a second location on the surface of the body that is spaced from the first location, and wherein the bio-impedance signal is induced within the body by the electrical signal injected from the first patch. In some embodiments, each of the plurality of patches comprises a ground electrode in electrical contact with the surface of the body. In some embodiments, the transmitter of the first patch is configured to inject an alternating current at a first frequency, the sensor of the second patch is configured to detect the bio-impedance signal from a voltage induced by the injected alternating current, the second patch comprises a transmitter configured to inject an alternating current at a second frequency that is different from the first frequency, and the first patch comprises sensor configured to detect a bio-impedance signal from the body that is induced by the alternating currents injected from the first patch and the second patch. In certain embodiments, the system further comprises a controller coupled to the second patch and configured to estimate the one or more physiological parameters based on the bio-impedance signal, wherein the one or more physiological parameters comprises a plurality of physiological parameters, and wherein the controller is configured to apply a source separation algorithm on the bio-impedance signal to separate a first physiological parameter from the plurality of physiological parameters. In certain embodiments, the controller is configured to adjust at least one of a frequency, an amplitude, and a shape of the electrical signal injected by the transmitter.

An embodiment of a method for monitoring one or more physiological parameters comprises (a) injecting an electrical signal into a body with a first patch positioned at a first location on a surface of the body, and (b) detecting a bio-impedance signal induced within the body by the injected electrical signal with a second patch positioned at a second location on the surface of the body and that is electrically isolated from the first patch, wherein the second location is spaced from the first location. In some embodiments, the method further comprises (c) independently electrically grounding the first patch and the second patch to the surface of the body. In some embodiments, the method further comprises (c) estimating the one or more physiological parameters based on the detected bio-impedance signal.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of exemplary embodiments of the disclosure, reference will now be made to the accompanying drawings in which:

FIG. 1 is a schematic representation of a resistor-capacitor circuit tissue model,

FIG. 2 is a schematic representation of an impedance mapping model;

FIGS. 3-5 are graphs illustrating data obtained from a simulation conducted using the impedance mapping model of FIG. 2;

FIG. 6 is a schematic representation of an embodiment of a system for monitoring physiological parameters in accordance with principles disclosed herein;

FIG. 7 is a schematic representation of an embodiment of a Bio-Z detecting patch of the system of FIG. 6 in accordance with principles disclosed herein;

FIG. 8 is a schematic representation of an embodiment of a signal injector unit of the patch of FIG. 7 according to some embodiments;

FIG. 9 is a schematic representation of an embodiment of a parameter extraction unit of the patch of FIG. 7 according to some embodiments;

FIG. 10 is a block diagram of an embodiment of a digital signal processing algorithm performable by the parameter extraction unit of FIG. 9 according to some embodiments; and

FIGS. 11-25 are graphs pertaining to data obtained from experiments of Bio-Z detecting patches.

### DETAILED DESCRIPTION

The following discussion is directed to various exemplary embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in the interest of clarity and conciseness. In addition, unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, as used herein, the terms “axial” and “axially” generally mean along or parallel to a given axis (e.g., central axis of a body or a port), while the terms “radial” and “radially” generally mean perpendicular to the given axis. For instance, an axial distance refers to a distance measured along or parallel to the axis, and a radial distance means a distance measured perpendicular to the axis. Further, when used herein (including in the claims), the words “about,” “generally,” “substantially,” “approximately,” and the like mean within a range of plus or minus 10%. The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

As described above, various techniques may be employed to monitor physiological parameters of a patient. Some techniques for monitoring physiological parameters are obtrusive to the patient. For instance, techniques that rely on PSG signals may require the usage of a belt that is securely attached to the patient, which may result in significant discomfort to the patient. As another example, capnography may require the patient to wear multiple nasal sensors or a face mask that also result in significant discomfort to the patient.

Additionally, wearable systems intended to provide an unobtrusive experience for the patient may have limited reliability in monitoring varying types of physiological parameters. For instance, PPG signals may have limited reliability for monitoring RR given that the mixing effect between the PPG signal and respiratory-induced variations of the vessels (relied on for monitoring RR using PPG signals) may be weak and dependent on skin thickness, temperature, sensor location, and other parameters. ECG-derived respiratory monitoring may also suffer from limited reliability.

Accordingly, embodiments disclosed herein include systems and methods for monitoring one or more physiological parameters using Bio-Z. Particularly, embodiments disclosed herein include systems for monitoring one or more physiological parameters that include a plurality of patches positionable at a plurality of locations on a surface of a body, wherein the plurality of patches are electrically isolated from each other. Additionally, each patch of the plurality of patches may be independently electrically grounded to the body. A first patch of the plurality of patches may comprise a transmitter configured to inject an electrical signal into the body at a first location on the surface of the body, and a second patch of the plurality of patches may be configured to detect a bio-impedance signal at a second location on the surface of the body, wherein the bio-impedance signal is induced by the electrical signal injected into the body from the first patch.

Further, embodiments disclosed herein include a method for monitoring one or more physiological parameters which includes injecting an electrical signal into a body with a first patch positioned at a first location on a surface of the body, and detecting a bio-impedance signal induced within the body by the injected electrical signal with a second patch positioned at a second location on the surface of the body and that is electrically isolated from the first patch, wherein the second location is spaced from the first location. The method may additionally include independently electrically grounding the first patch and the second patch to the surface of the body, and/or estimating the one or more physiological parameters based on the detected bio-impedance signal.

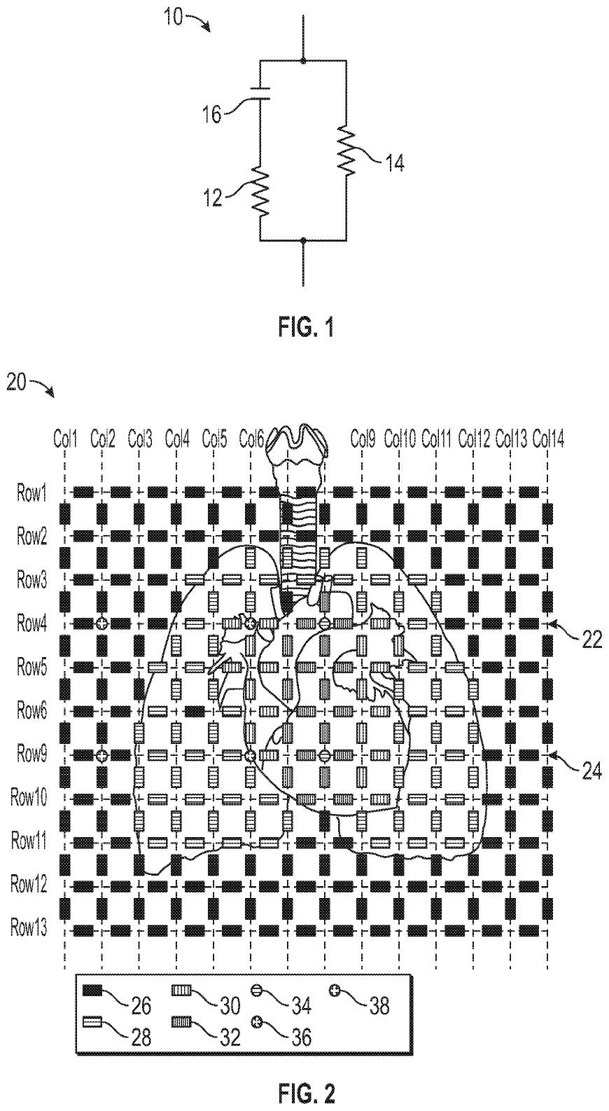

Through establishing electrical contact with the skin via electrodes, it is possible to stimulate a patient's epidermis with an electrical signal, such as an AC signal, which seeks the last impeding path through the tissues of the patient. At high frequencies, the AC signal may pass through a combination of extracellular fluid, cell membrane, and intracellular fluid, thereby capturing a mixture of information about the physiological status of the patient. Referring to FIG. 1, the interaction between the AC signal and the tissue of the patient can be modeled as a resistor-capacitor (RC) circuit 10 as shown in FIG. 1. Particularly, by representing the intracellular and extracellular fluids with resistors, 12 and 14 respectively, and the cell membrane as a capacitor 16 positioned in parallel to the extracellular resistor 14 an in series with the intracellular resistor 12.

In this configuration, the frequency of the injected AC signal directly affects the impedance path. Particularly, at low frequencies, the AC signal may not be able to penetrate through the membrane capacitor 16 and thus may generally travel only through the extracellular fluid, whereas at very high frequencies the tissue RC circuit 10 will act as a low-pass filter (LPF) allowing high-frequency AC signals to penetrate the cell membrane and travel through the intracellular fluid contained therein. In the calculation of a transfer function from a cellular region, the ratio of RC parameters with respect to each other may be more significant than the values themselves.

Several advantages may be obtained from injecting a high-frequency electrical signal, such as an AC signal, into the epidermis of a patient. First, increasing frequency significantly decreases the electrode-to-skin impedance caused by the unideal current transfer between ions and electrons. Therefore, the voltage drop across the electrodes at the location of injection becomes minimal allowing higher amplitudes of current stimulation. Second, the allowance of injection amplitude before damaging the tissue increases may increase for frequencies approximately between 1 kilohertz (kHz) and 100 kHz. Third, changes in the impedance of the tissues and underlying cells due to variations in physiological parameters (blood flow, lung and heart movements, hydration, and muscle movements, etc.) may be more reliably carried by higher frequency AC signals. Thus, flicker noise which may dominate the low-frequency AC spectrum will have less of an impact on information carried by a high-frequency AC signal.

Variations in a Bio-Z signal based on the physiological autonomic motive forces (e.g., heart, lungs, pulsatile movement, etc.) as well as controlled muscle forces (respiration based chest motion, stretching, etc.) may be detected by injecting or passing an electrical signal through the upper chest of the patient. Particularly, as the injected electrical signal travels through the upper chest, the electrical signal may be modulated by physiological autonomic and controlled muscle forces of the patient. One or more sensors positioned on the upper chest of the patient may measure or detect the modulated electrical signal as the electrical signal is simultaneously injected into the patient. Thus, placing multiple sensors at various positions on the patient's body may capture these activities from multiple locations with various contributions of each activity. By capturing the autonomic motive forces and controlled muscle forces of the patient at a plurality of locations on the patient's body, the application of source separation techniques may be employed to separate these individual activities into individual estimated physiological parameters.

Referring to FIGS. 2-5, as an exemplary illustration of this methodology, an impedance mapping model 20 of an upper chest of a patient is shown in FIG. 2. Chest impedance model 20 simulates variations caused by the injection of an AC signal (positive injection point identified by arrow 22 in FIG. 2 and the negative injection point identified by arrow 24), which may be matched with experimental data. In simulations employing model 20, the time constant of the tissue impedance is generally smaller than body impedance changes due to heart and lung activities (i.e., τ≈μs<<τₕₑₐᵣₜ/lungs≈s). The difference in the time constants between tissue impedance and body impedance changes may be due to the values of membrane capacitor 16, intracellular resistor 12, and extracellular resistor 14 of the tissue RC circuit 10 being on the order of nano-farads and tens of ohms, respectively. Therefore, the complexity of chest impedance model 20 may be minimized by replacing the tissue RC circuit 10 with a resistive element. With this simplification, the fastest frequency component of chest impedance model 20 may be due to time variant resistance (i.e. 1.3 Hz) instead of frequency of the injected AC signal (10 kHz in this example). Hence, the Nyquist sampling criterion requirement may be relaxed by approximately five orders of magnitude, allowing a larger time step selection for the simulation and a substantial reduction in the number of samples per second.

Chest impedance model 20 generally comprises a resistive 10-by-13 matrix (130 resistive elements in total) to simulate an entire upper chest of a patient. Particularly, the 130 resistive elements comprise a plurality of body-body tissue resistive elements 26, a plurality of lung-body tissue resistive elements 28, a plurality of heart-lung tissue resistive elements 30, and a plurality of heart-body tissue resistive elements 32, where tissue resistive elements 26-32 are arranged in the 10-by-13 matrix to model the location of the heart, lungs, and body tissues of an upper chest of a patient.

Each tissue resistive element 26-32 of chest impedance model 20 corresponds to two parallel resistive tissue blocks each having an assigned resistance value to include the possibility of representing regions with multiple organs (i.e. heart and lungs) associated with a single element. For example, each heart-lung resistive element 30 may comprise a modeled heart resistor positioned in parallel with a modeled lung resistor while each lung-body tissue resistive element 28 may comprise the modeled lung resistor positioned in parallel with a modeled body tissue resistor. Each resistive tissue block of each tissue resistive element 26-32 is assigned to have a non-stationary part that is approximately five orders of magnitude larger than the time-dependent part to match experimental results, as will be discussed further herein. Chest impedance model 20 also includes a plurality of differential voltage sensors 34, 36, and 38 positioned at different locations within the 10-by-13 matrix and configured to detect a Bio-Z signal from a voltage induced by the injection of an AC signal into the 10-by-13 matrix at the injection point indicated by arrows 22, 24.

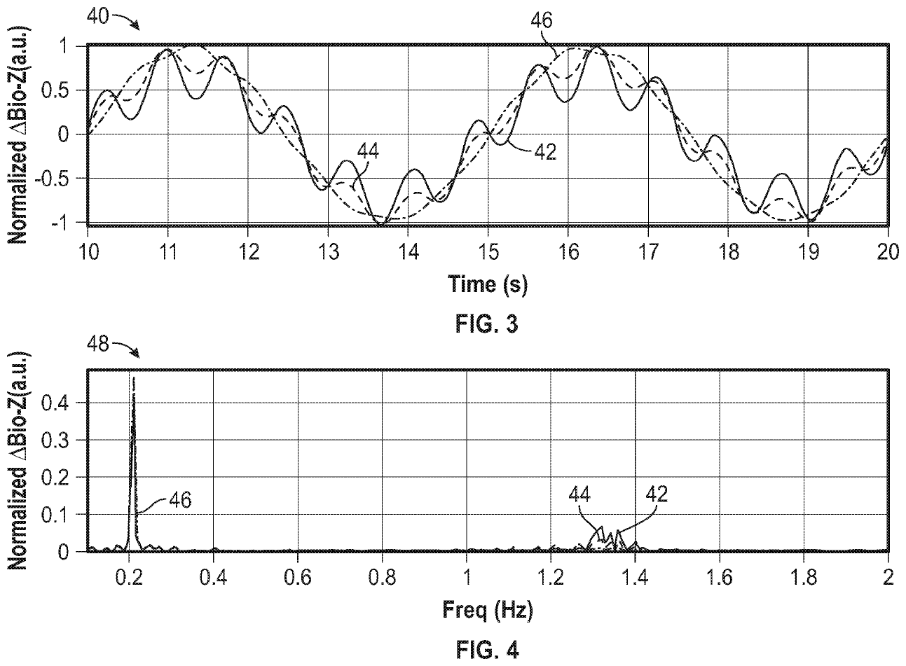

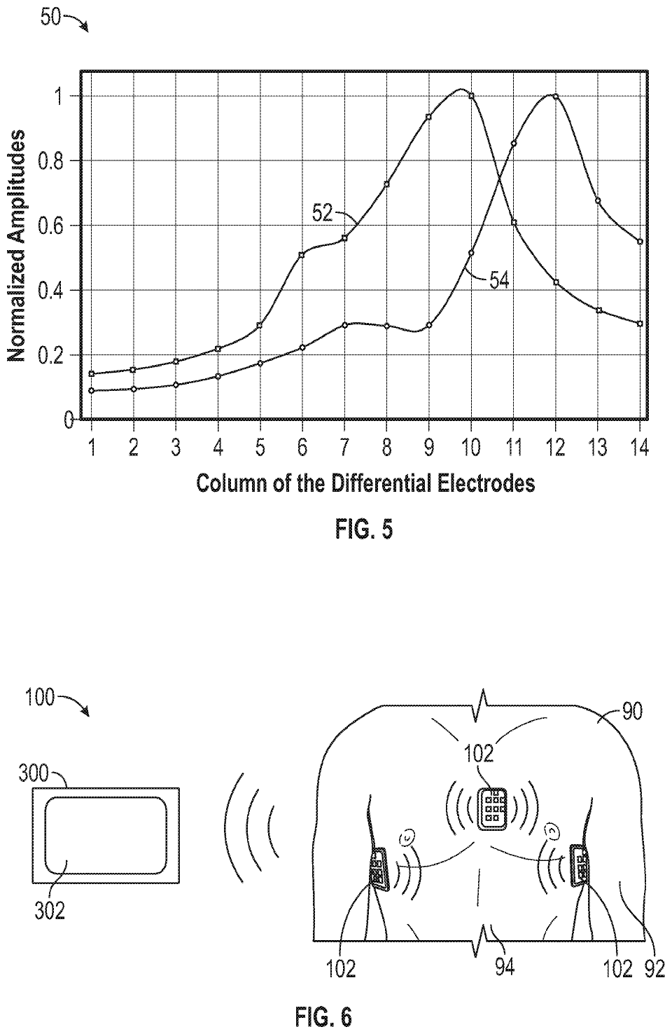

An exemplary simulation was performed using the chest impedance model 20 where the body tissue block of each body-body resistive element 26, long-body resistive element 28, and heart-body resistive element 32 comprised approximately 10⁴ micro-ohms (mΩ), the heart tissue block of each heart-lung resistive element 30 and each heart-body resistive element 32 comprised approximately (10⁴+25 sin(2π*1.3(t)) mΩ where (t) comprises simulation time, and the lung tissue block of each lung-body resistive element 28 and heart-lung resistive element 30 comprised approximately (10⁴+50 sin(2π*0.2(t)) mΩ. FIGS. 3, 4 comprise graphs 40, 48 which illustrate Bio-Z normalized sensor readings (in arbitrary units (a.u.)) 42, 44, and 46 (corresponding to sensors 34, 36, and 38, respectively) in the time and frequency domains, respectively, produced from the simulation. Graphs 40, 48 indicate that each sensor 34, 36, and 38 was able to capture respiratory activity (occurring generally at 0.15 Hz), no matter the location of each sensor 34, 36, and 38. However, sensor 38 showed a relatively weak response to the variations caused by heart activity (occurring generally at 1.3 Hz) both due to the location of sensor 38 with respect to the heart and the injection point indicated by arrows 22, 24. FIG. 5 comprises a graph 50 that illustrates the normalized amplitude of the simulated pulse 52 (occurring generally at 1.3 Hz) and respiration (occurring generally at 0.2 Hz) produced from the simulation across the columns of the 10-by-13 matrix of chest impedance model 20. For this simulation, only the columns of the sensors were changed, whereas the rows of the differential points of the voltage sensors 34, 36, and 38 remained unchanged at rows 4 and 9 as shown in FIGS. 3, 4. The results of the simulation indicate that the peaks for simulated heart activity and lung activity do not necessarily need to happen at the same sensing point, justifying placing multiple patches across different locations. Hence, chest impedance model 20 indicates that a holistic set of observations of the patient's physiological parameters may be directly from a plurality of spaced-apart differential voltage sensors. This sensor arrangement also allows for the creation of a diverse mixing matrix, where although the exact locations of the sources of the physiological parameters are unknown, by leveraging a source separation algorithm, it is possible to estimate these physiological parameters. Moreover, chest impedance model 20 indicates that although the exact values of the placement of sensors 34, 36, and 38, as well as the current flowing through each resistive element 26-32, may also remain unknown, the impedance variations still appear at the voltage sensors 34, 36, and 38. Thus, chest impedance model 20 demonstrates the sources (physiological parameters) which may appear in the sensed Bio-Z at different locations of the patient's upper chest.

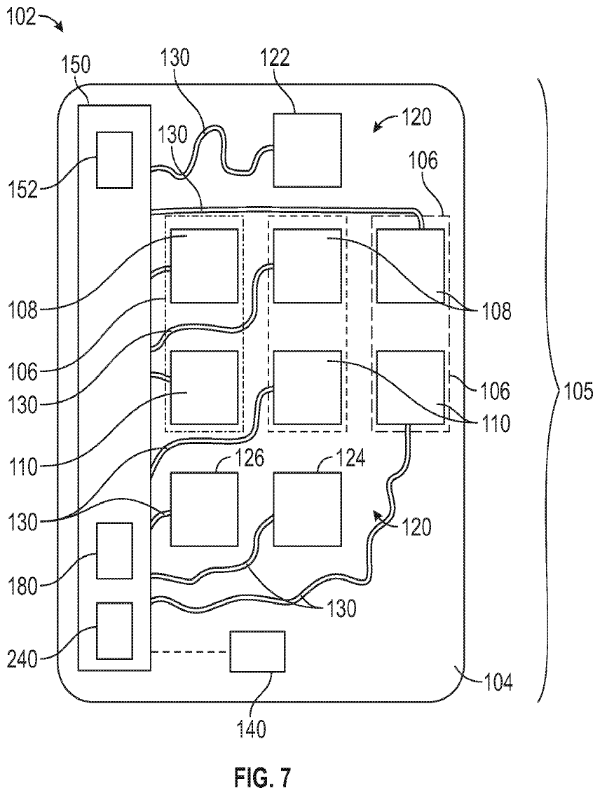

Referring to FIGS. 6, 7, an embodiment of a system 100 for monitoring one or more physiological parameters of a patient 90 using Bio-Z is shown in FIGS. 6, 7. System 100 generally includes a plurality of wirelessly communicable, Bio-Z sensing patches 102 positionable on or configured to releasably couple to the surface or skin of the patient 90. Each patch 102 is generally configured for injecting a programmable electrical signal, such as an AC and/or alternating voltage signal, into the body 92 of patient 90, and sense or detect a Bio-Z signal from the body 92 that is induced by the injected AC signal.

In the embodiment shown in FIGS. 6, 7, each patch 102 is positioned on the upper chest 94 of the patient's body 92. Particularly, a pair of the patches 102 may be positioned underneath each armpit of the patient 90 while a third patch 102 may be positioned across the patient's heart (left the sternum). In this configuration, patches 102 may provide full or global coverage of the patient's entire upper chest 94 to thereby permit the monitoring of global variations in physiological parameters (e.g., variations directly correlated with heart and respiratory activity of the patient 94) of the patient 90 rather than only localized variations (e.g., localized to the wrist, ankles, arms, etc. of the patient 90) observed via a single sensor which may only be partially correlated to respiratory-induced blood flow of the patient 90, making such localized observations sensitive to motion artifacts and the placement of the sensor on the patient's body 92. However, the placement of patches 102 on the patient 90 may vary depending on the application. Additionally, while in this embodiment system 100 includes three patches 102 positioned on the patient 90, in other embodiments, the number of patches 102 of system 100 may vary. For example, system 100 may comprise a single patch 102 or more than three patches 102 positioned at locations other than the upper chest 94 of the patient 90.

To ensure that patches 102 remain comfortable to wear by the patient 90, patches 102 are separated and electrically isolated from one another, thereby giving up the phase calibration between the patches 102. Given that patches 102 do not include a common potential, only the frequency information is known by each patch 102. In some embodiments, this information is sufficient to carry out a phase insensitive lock-in based amplitude demodulation to fully capture variations in the Bio-Z signal sensed by patches 102. The variations in the received Bio-Z signal may correspond to the physiological signals or parameters initiated by the periodic heart and lung movements of patient 90.

System 100 may also include an input/output (I/O) unit 300 in electronic wireless communication with one or more of the patches 102 of system 100. I/O unit 300 may include a screen or display 302 for viewing signals received from one or more of the patches 102. For instance, the display 302 may display one or more continuously updated physiological parameters of the patient 90 estimated by the patches 102 of system 100. In some embodiments, the display 302 of I/O unit 300 may display physiological parameters of the patient 90 estimated by patches 102 in real-time or near real-time. For instance, in some embodiments, the delay between the sensing of the patient's Bio-Z by patches 102 and the display of one or more estimated physiological parameters derived from the sensed Bio-Z may be less than one second, such as approximately 100 milliseconds; however, in other embodiments the delay between the sensing of the patient's Bio-Z and the display of one or more estimated physiological parameters may vary.

As shown particularly in FIG. 7, each patch 102 may generally include a body or adhesive pad 104 and a plurality of electrical components coupled to and positioned on the adhesive pad 104, including a plurality of sensors 106, a transmitter 120, a ground (GND) electrode 126, a battery 140, and a control unit or controller 150. In some embodiments, the adhesive pad 104 of each patch 102 may have a maximum size or length 105 of approximately three inches (in); however, the size of pad 104 may vary. For instance, the maximum size or length 105 of each pad 104 may vary approximately between 1 in and 5 in.

In some embodiments, each patch 102 of system 100 may be configured similarly with similar components. However, in other embodiments, some patches 102 may vary in configuration from others. For example, in some embodiments, one or more patches 102 may comprise a transmitter 120 but not a sensor 106 while one or more other patches 102 comprise one or more sensors 106 but do not include a transmitter 120. In other words, in some embodiments, system 100 may comprise at least one patch configured to inject an AC signal into the patient 90 but not to sense Bio-Z from the patient 90 and one or more patches configured to sense Bio-Z but not to inject the AC signal into the patient 90. Additionally, some patches 102 may comprise a single senor 106 while other patches 102 may comprise a plurality of sensors 106. Further, the controllers 150 of each patch 102 may also vary in configuration.

Transmitter 120 may comprise a current transmitter configured to inject an AC signal at a programmable frequency, amplitude, and/or shape and sensors 106 may comprise voltage sensors generally configured to detect a Bio-Z signal from the patient 90 based on a voltage induced by the current injected into the patient 90 by the transmitter 120 of one of the patches 102. However, in other embodiments, transmitter 120 may be configured to inject an alternating voltage signal into the body 92 of the patient 90 and each sensor 106 may comprise a current sensor configured to detect a Bio-Z signal from the patient 90 based on a current induced by the injected voltage.

In this embodiment, at least one patch 102 of system 100 comprises a plurality of sensors 106. The plurality of sensors 106 are located close proximity and may provide redundant information to mitigate the effect of noise due to motion of the patient 90 and improve signal quality and SNR by combining the output of each sensor 106. Particularly, noise due to the motion of the patient 90 may be mitigated given that the noise introduced in the measurement of each sensor 106 will be unique given that each sensor 106 is positioned at a different location on the patient's body 92.

At least one patch 102 of system 100 comprises a transmitter 120. In some embodiments, a plurality of patches 102 comprise transmitters 120 for independently injecting a plurality of electrical signals (e.g. AC current signals) simultaneously into the patient's body 92 as a plurality of sensors 106 of system 100 simultaneously detect Bio-Z signals from the patient's body 92 induced by the injection of the plurality of electrical signals. Each transmitter 120 of system 100 may inject an electrical signal at a unique, programmable frequency into the patient's body 92 to allow for the separation of Bio-Z signals detected by the sensors 106 of system 100.

While in this embodiment each patch 102 comprises three separate sensors 106, in other embodiments, the number of sensors 106 may vary. For instance, each patch 102 may comprise a single sensor 106 or more than three sensors 106. Each sensor 106 may include a positive electrode and a negative electrode 110 while the transmitter 120 comprises a positive electrode 122 and a negative electrode 124. The electrodes 108, 110, 122, 124, and 126 may be coupled or placed in signal communication with controller 150 via electrical tracing 130 extending between each electrode 108, 110, 122, 124, 126 and the controller 150. At least some of electrodes 108, 110, 122, 124, 126 may comprise pre-gelled silver chloride (Ag/AgCl) electrodes having a diameter of approximately between 20 millimeters (mm) and 25 mm, however, the configuration of each electrode 108, 110, 122, 124, 126 may vary. Separating the sensing electrodes 108, 110 and transmission electrodes 122, 124 may leverage the four-point sensing mechanism to mitigate the effect of the electrode-skin interface. This is because the sensing electrodes may be followed by a buffer or instrumentational amplifier (IA) with a very high input gain rejecting a current flow to its input. Therefore, the voltage drop across the sensing patches caused by a portion of the injected current that is passing through the underlying Bio-Z may be buffered to the output as a detected Bio-Z signal with negligible loss and interference.

Without needing to commonly ground patches 102 (e.g., via a common hardwired connection between each patch 102), a reference point to the differential voltage inputs (e.g., the inputs provided by each sensor 106) of each patch 102 may be provided by individually and separately grounding each patch 102 to the body 92 of patient via the GND electrode 126 of each patch 102. By separately grounding each patch 102 to the patient's body 92, the common-mode rejection ratio may be improved by preventing the amplification of the floating common-mode signals and increasing the received signal to noise ratio (SNR) at the sensors 106. Patches 102 of system 100 are electrically isolated from each other. As used herein, the term “electrically isolated” means that no direct electrical communication (wireless or wired) takes place between each patch 102. Instead, the only communication which occurs between each patch 102 is the communication mediated by the body 92 of the patient 90. Particularly, the only communication which takes place between the patches 102 of system 100 is the detection of a Bio-Z signal (via one or more of the sensors 106 of one of the patches 102) from the patient's body 92 induced by the electrical signal (e.g., an AC current signal and/or an alternating voltage signal, etc.) injected into the patient's body 92 (via the transmitter of one of the patches 102).

The battery 140 of each patch 102 may be generally configured to electrically power the transmitter 120 and controller 150 of the patch 102. Battery 140 may be configured to support several hours of continuous operation of the patch 102. In some embodiments, battery 140 may comprise a lithium polymer (LiPo) battery; however, the configuration of battery 140 may vary.

Controller 150 is generally configured to control the operation of the sensors 106 and transmitter 120 of the patch 102. Controller 150 may comprise a singular controller or control board or may comprise a plurality of controllers or control boards that are coupled to one another. Controller 150 may comprise one or more flexible printed circuit boards (PCB) and/or one or more rigid PCBs with flexible or rigid connections therebetween. For convenience, and to simplify the drawings, controller 150 is depicted schematically in FIG. 7 as a single controller unit that is coupled to various components of patch 102 and positioned on adhesive pad 104; however, in some embodiments, at least some components of controller 150 may not be positioned on adhesive pad 104 and instead may be positioned distal adhesive pad and in signal communication (e.g., wired or wireless communication) with components of controller 150 positioned on adhesive pad 104. Controller 150 may comprise at least one processor and associated memory. The one or more processors (e.g., microprocessor, central processing unit (CPU), or collection of such processor devices, etc.) of controller 150 may execute machine-readable instructions provided on the memory (e.g., non-transitory machine-readable medium) to provide controller 150 with all the functionality described herein. The memory of controller 150 may comprise volatile storage (e.g., random access memory (RAM)), non-volatile storage (e.g., flash storage, read-only memory (ROM), etc.), or combinations of both volatile and non-volatile storage. Data consumed or produced by the machine-readable instructions of controller 150 can also be stored on the memory thereof. As noted above, in some embodiments, controller 150 may comprise a collection of controllers and/or control boards that are coupled to one another. As a result, in some embodiments, controller 150 may comprise a plurality of the processors, memories, etc.

In this embodiment, controller 150 generally includes a signal injector unit 152, a parameter extraction unit 180, and a wireless transmitter 240. In this embodiment, each unit 152, 180, and the wireless transmitter 240 of controller 150 may be located directly on the adhesive pad 104 of patch 102. However, in other embodiments, certain components of controller 150 may be spaced from the adhesive pad 104. For instance, all or some of the components of injector unit 152 and/or parameter extraction unit 180 may be located distal patch 102, such as within I/O unit 300 or another electronic device in signal communication with I/O unit 300. In embodiments were all or at least some components of units 152, 180 are positioned distal patch 102, signals may be transmitted between patch 102 and the distal components via a hardwired or wireless (e.g., via wireless transmitter 240) signal link.

The signal injector unit 152 of controller 150 is generally configured to generate a programmable AC signal for injection into the body 92 of patient 90 via the transmitter 120 of patch 102. Particularly, signal injector unit 152 of controller 150 may be configured to adjust the frequency, amplitude, and/or shape (e.g., sinusoidal, square, saw tooth, triangle wave, etc.) of the AC signal. For example, the signal injector unit 152 of a controller 150 of one of the patches 102 may adjust a shape of the injected AC signal from a sinusoidal shape to a square or triangle shape, etc. As the AC signal passes through the upper chest 94 of the patient 90, the AC signal induces a voltage measured by the sensors 106 of the patches 102 positioned at different locations on the patient's body 92.

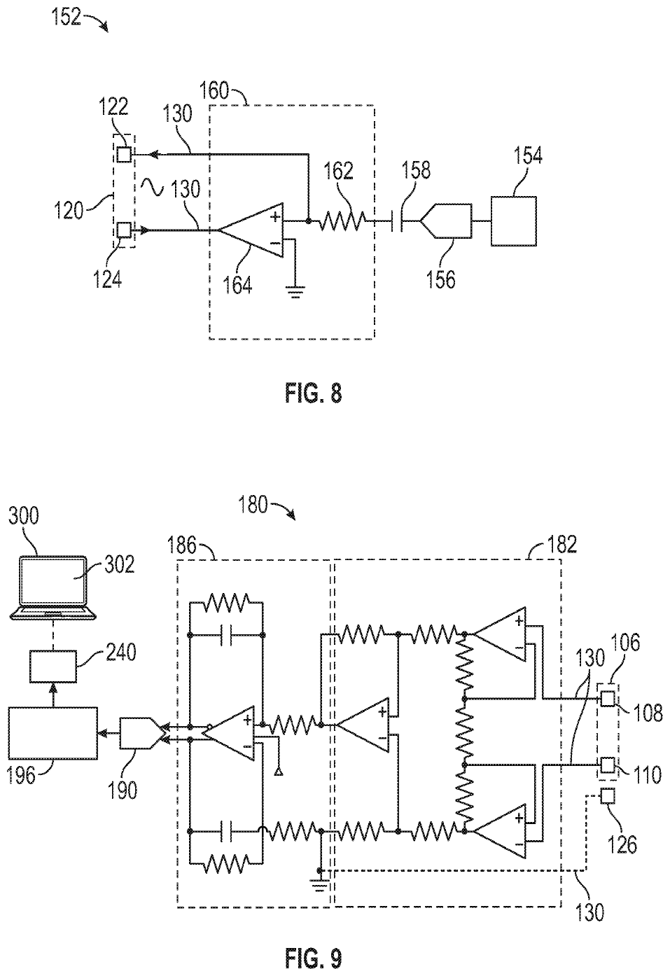

Referring to FIG. 8, a schematic representation of an exemplary signal injector unit 152 of the controller 150 of FIG. 7 is shown in FIG. 8. FIG. 8 may only illustrate some of the components of signal injector unit 152, and some embodiments of signal injector unit 152 may be configured differently from the unit 152 shown in FIG. 8. Signal injector unit 152 may generally include an injector microcontroller 154, a digital-to-analog converter (DAC) 156, an injector capacitor 158, and a voltage-to-current converter (V-to-I) 160. Injector microcontroller 154 is generally configured for operating the DAC 156 to generate an AC signal with a programmable frequency, amplitude, and/or shape. In some embodiments, injector microcontroller may comprise a 32-bit ARM® Cortex®-M4F microcontroller available from Texas Instruments.

DAC 156 of signal injector unit 152 may be 16-bit and may generate an AC signal having an output amplitude of approximately between 5.0 Volts (V) to 5.5 V providing a resolution of approximately 60 micro-Volts (pV) to 90 pV. Injector capacitor 158 may be positioned at the outlet of DAC 156 to prevent the injection of direct current (DC) into the skin of patient 90. V-to-I is generally configured to convert the alternating voltage signal outputted by DAC 156 into an AC signal and may include a resistor 162 fed to the negative input of a low power precision amplifier 164 with the positive input being grounded. The amplifier 164 may support low power applications with a typical supply current of approximately between 3.0 milliamps (mA) and 4.0 mA. Electrodes 122, 124 of transmitter 120 may be connected to the feedback loop of amplifier 164, allowing a root means square (RMS) current of approximately between 0.5 mA to 1.0 mA.

The parameter extraction unit 180 of controller 150 is generally configured to sense Bio-Z signals induced by the injection of an AC signal into the patient's body 92 via signal injector unit 152 and extract one or more estimated physiological parameters of the patient 90 derived from the sensed Bio-Z signals. The amplitude of the variations in the Bio-Z signals due to blood flow is very small (e.g., approximately between 10 mΩ and 200 mΩ) with several inches of separation between electrodes 108, 110 of the sensor 106. Thus, parameter extraction unit 180 may comprise low-noise Bio-Z sensing hardware using discrete components.

Particularly, referring to FIG. 9, a schematic representation of an exemplary parameter extraction unit 180 of the controller 150 of FIG. 7 is shown in FIG. 9. FIG. 9 may only illustrate some of the components of current extraction unit 180, and some embodiments of parameter extraction unit 180 may be configured differently from the unit 180 shown in FIG. 9. Parameter extraction unit 180 may generally include a low power instrumentation amplifier 182, a LPF 186, an analog-to-digital converter (ADC) 190, and an extraction microcontroller 196.

Amplifier 182 of parameter extraction unit 180 may provide a gain of approximately 20 decibels (dB) with approximately a 110 dB common-mode rejection ratio. The LPF 186 of parameter extraction unit 180 may comprise an anti-aliasing filter having a cut-off frequency (e.g., approximately 30 kHz) based on the sampling frequency of the ADC 190. ADC 190 may sample three independent Bio-Z channels (e.g., the Bio-Z signal detected by each sensor 106 of patch 102) simultaneously with 24-bit resolution. A single multi-channel ADC 190 may be used to carry out all analog to digital conversion to isolate the processing of each individual analog channel. The multi-channel digital output of the ADC 190 may be provided to the extraction microcontroller 196 of parameter extraction unit 180 for digital signal processing (DSP). In some embodiments, the parameter extraction unit 180 may be configured for acquiring Bio-Z measurements (provided by sensors 106) with a RMS error of less than 1 mΩ, which is significantly lower than Bio-Z variations due to variations in blood flow (approximately 50 mΩ).

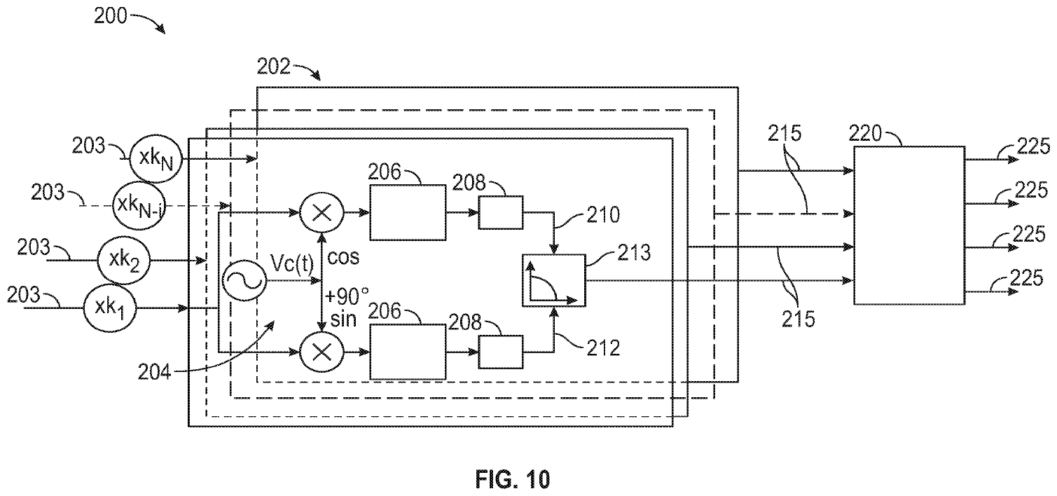

Extraction microcontroller 196 of parameter extraction unit 180 may perform DSP on the digital output of ADC 190 prior to estimating one or more physiological parameters of the patient 90 based on the output of ADC 190. For example, referring to FIG. 10, a block diagram of an embodiment of a DSP algorithm 200 is shown in FIG. 10. DSP algorithm 200 may generally include a filtering or demodulation algorithm 202 followed by a parameter separation algorithm 220. In some embodiments, both the demodulation algorithm 202 and parameter separation algorithm may be stored on a memory and performed by a processor located directly on path 202; however, in other embodiments, at least some of the steps comprising demodulation algorithm 202 and/or parameter separation algorithm 220 may be stored on a memory and performed by a processor of controller 150 that is not positioned on the adhesive pad 104 of any of the patches 102 of system 100 (e.g., these steps may be performed by a processor of controller 150 that is spaced from each patch 102 and in communication with one or more patches 102 via a wireless or hardwired connection).

In some embodiments, demodulation algorithm 202 may receive a plurality of digital raw input signals 203 each corresponding to an output from one of the channels of the ADC 190 of parameter extraction unit 180. In other words, each raw input signal 203 corresponds to a digitized Bio-Z signal detected by one of the sensors 106 of a patch 102. Demodulation algorithm 202 is generally configured to filter or demodulate the received raw input signals 203 to produce a plurality of corresponding demodulated signals 215 to minimize image noise in each demodulated signal 215 prior to feeding the demodulated signals 215 the parameter separation algorithm 220.

In some embodiments, demodulation algorithm 202 may receive information pertaining to the unique frequency of each electrical signal injected into the patient's body 92 by each transmitter 120 of system 100, and demodulation algorithm 202 may utilize this frequency information when producing demodulated signals 215. For example, if a first transmitter 120 of a first patch 102 of system 100 injects a first electrical signal at a first frequency into the patient's body 92 while simultaneously a second transmitter 120 of a second patch 102 of system 100 injects a second electrical signal at a second frequency into the patient's body 92 demodulation algorithm 202 may receive as an input information pertaining to the first and second frequencies of the first and second electrical signals injected into the patient's body 92. The demodulation algorithm 202 may include different bandpass filters centered around the first and second frequencies to separate the signal injected by the first transmitter 120 from the signal injected by the second transmitter 120 to produce a first demodulated signal 215 corresponding to the first frequency and a second demodulated signal 215 corresponding to the second frequency.

In some embodiments, demodulation algorithm 202 may comprise a quadrature demodulation technique or algorithm. For example, after being multiplied by a channel constant 205 (having units of ON), each input signal 203 may be demodulated via a lock-in based demodulator 204 to produce a pair of signals which may pass through a LPF 206 (e.g., a Butterworth LPF) to reject image noise as well as high high-frequency fluctuations while permitting heart rate signals up to 180 beats per minute (bpm). In some embodiments, each LPF may have a cut-off of approximately 4.4 Hz. After passing through LPFs 206, the pair of signals may be down-sampled at block 208 to produce an in phase (I) signal 210 and a quadrature (Q) signal 212 which may be combined at block 214 to produce one of the demodulated signals 215 (e.g., the square root of the sum of the squared in phase signal 210 and the squared quadrature signal 212 may be taken to produce the demodulated signal 215) that is inputted to the parameter separation algorithm 220. In some embodiments, prior to combination at block 214, the pair of signals may each be down-stepped at block 208 from a frequency of approximately between 90 kHz to 100 kHz to a frequency approximately between 350 Hz and 400 Hz.

In other embodiments, demodulation algorithms as well as other filtering algorithms may be used in place of the quadrature algorithm described above for producing demodulated signals 215. For example, in some embodiments, an envelope detection algorithm may be employed to condition raw input signals 203 prior to feeding the signals to the parameter separation algorithm 220. Additionally, in some embodiments, raw input signals 203 may be fed directly to parameter separation algorithm 220.

In some embodiments, an ECG signal may be obtained from one of the patches 102 of system 100. Particularly, a second-order LPF (e.g., a Butterworth LPF) may be applied to one of the raw input signals 203. In the LPF may have a cut-off frequency of approximately 30 Hz.

The demodulated signals 215 outputted from demodulation algorithm 202 may each comprise a plurality of physiological parameters of the patient 90 (e.g., HR, RR, BP, etc.) which may be separated via the parameter separation algorithm 220 to provide a plurality of signals corresponding to estimated physiological parameter signals 225 of the patient 90. In some embodiments, parameter separation algorithm 220 may comprise a blind source separation (BSS) technique or algorithm configured to separate the signals sourced independently by heart and lungs from each other using the multiple simultaneous observations (e.g., the plurality of demodulated signals 215 inputted to the parameter separation algorithm 220).

Particularly, in some embodiments, parameter separation algorithm 220 may comprise a second-order blind identification (SOBI) algorithm which leverages information on the second-order statistics of the Bio-Z observations and prior knowledge of human physiology to separate and extract parameters corresponding to, for example, heart and respiration rates. A SOBI algorithm may be particularly adapted for using the time coherence of the physiological parameter signals appearing in the observations (e.g., demodulated signals 215) with different time delays. Additionally, a SOBI algorithm may be robust to the time delay variations of the physiological parameters for each observation. The unknown delays may be introduced due to blood flow being present at various parts of the upper chest 94 but with different phases and the capacitive components of the Bio-Z sensing.

In other embodiments, source separation algorithms other than SOBI may be utilized, such as, for example, Independent Component Analysis (ICA). However, Bio-Z measurements over a large chest area may demonstrate high variances in terms of phase delays given blood arrives at different time instances to each sensing location (e.g., the location of each patch 102 on the upper chest 94 of the patient 90), which may limit the utility of ICA and other purely statistical methods. Conversely, Second Order Separation (SOS) techniques or algorithms, such as SOBI, contrast with this feature of statistical methods, where the separation takes place due to the temporal characteristics in the ongoing activity of the underlying physiological parameters. In order to enhance the robustness of the SOS algorithms and combat the ambiguity in the time delays introduced by the patient's body 92, the SOBI algorithm utilized in this embodiment may be run iteratively multiple times with a set of preselected time lags introduced to the demodulated signals 215 prior to each run. Given that the SOBI algorithm is based on joint diagonalization of multiple covariance matrices having different time delays, instead of a single unique matrix utilized in other SOS techniques, the SOBI algorithm may improve the robustness in separating the heart and lung sources at low processing cost.

To briefly outline an exemplary SOBI algorithm which may comprise the source separation algorithm 220, input data can be represented as a vector of N recorded signals (e.g., demodulated signals 215), v=[v₁, v₂, . . . , v_{N}]^{T}, as the observations of M unknown independent physiological parameters or sources represented as, s=[s₁, s₂, s_{M}]^{T}. Not intending to be bound by any particular theory, the instantaneous linear mixture can be modeled as shown in the equation below, where A comprises an n×m mixing matrix, n(t) comprises additive noise, v(t) comprises vector of recorded signals (e.g., demodulated signals 215) referenced above, and s(t) comprises the vector of independent physiological parameters or sources referenced above:

v(t)=A×s(t)+n(t) (1)

In Equation (1), n(t) represents the additive noise modeled under two assumptions: source signals s(t) and noise n(t) are statistically independent, and n(t) is white, stationary and with zero mean. SOS-based source separation techniques may leverage the temporal correlations between the observations. Hence, in some applications, SOS-based source separation techniques may provide an advantage for scenarios where the observed signals retain low SNR given that the noise may not be common between the observations and may not significantly impact the correlation estimations. In addition, noise elements that are not Gaussian can be added to the source estimation problem.

The SOBI algorithm may only use the array of v(t) observations, without any prior knowledge of the model, to find the mixing matrix A and obtain the estimated and uncorrelated physiological parameters or sources represented as, y=ŝ. As described further below, and not intending to be bound by any particular theory, in order to obtain the mixing matrix A, an exemplary SOBI algorithm may begin with process of whitening the observations to reduce the determination of the n×m mixing matrix A to a unitary m×m matrix, U, without any loss of generality, using the whitening matrix, W, in accordance with the equation presented below:

A=W^{H}×Û (2)

Not intending to be bound by any particular theory, within a preset time delay window (τ), the exemplary SOBI algorithm may first calculate a set of covariance or correlation matrices in accordance with Equations (3) and (4) presented below, where z(t) comprises the whitened raw Bio-Z signals (e.g., whitened input signals 203), and τ comprises the time lag:

z(t)=W×v(t) (3)

for j=1 to τ do: R_{z}[j]=E{z(;,j:N)×z(:,1:N−j)^{H}} (4)

In some embodiments, τ may comprise 100, corresponding to a ±13 millisecond (ms) time window, with a sampling frequency of 375 Hz following the down-stepping performed at the demodulation algorithm 202; however, in other embodiments, τ may vary.

Not intending to be bound by any particular theory, following the calculation of the covariance matrix (e.g., in accordance with Equations (3), (4) presented above), a joint diagonalization analysis may be performed to find the orthonormal change of basis. At the output, the matrix with the highest sum-squared cross-correlation value may be selected as the first estimated component, and the iteration may continue until all m components are determined. For example, a joint diagonalization Û of {R_{z}[kⱼ]|j=1, . . . , τ} with the minimum sum-squared off diagonals may be determined. Following the determination of joint diagonalization U, mixing matrix A may be determined in accordance with Equation (2) presented above.

Not intending to be bound by any particular theory, estimated physiological sources or parameters (e.g., sources or parameters corresponding to physiological parameter signals 225) may be calculated in accordance with Equation (5) presented below, where y(t) comprises an estimated physiological source or parameter:

y(t)=A×v(t) (5)

Bio-Z signals provided by patches 102 positioned across the upper chest 94 of the patient 90 may provide a strong reflection of the respiration of the patient 90. This reflection may be captured from each sensor 106 of each patch 102 simultaneously. In addition, each sensor 106 may capture a set of other internal and external sources such as heart movements, blood flow, motion artifacts and other physiological parameters of the patient 90 which do not necessarily appear with the same temporal structure in the Bio-Z signals detected by the sensors 106. Due to the strong appearance of the respiration cycle in the temporal characteristics of the detected Bio-Z signals, the first estimated physiological parameter signal 225 at the output of SOBI algorithm that has the highest eigenvector may comprise a respiratory signal of the patient 90 (e.g., BioZR(t)=y₁(t)) from which various physiological parameters pertaining to the respiration of the patient 90 may be derived.

In some embodiments, for the physiological parameter signal 225 corresponding to the respiration signal of the patient 90, an additional LPF (e.g., a second-order Butterworth low-pass filter having approximately a 1 Hz cut-off) may be applied to remove high-frequency oscillations, followed by a peak detection algorithm to calculate inter-breath intervals (IBrI) of the patient 90. Optionally, in order to provide an average estimate of the RR of the patient 90 for a preset interval, a 60-second averaging window may be applied to the calculated IBrIs with approximately 55 seconds of overlap.

In addition to the estimated physiological parameter signals 225, the exemplary SOBI algorithm may estimate the mixing matrix A (e.g., via Equation (2) presented above), which allows for the isolation of each physiological parameter of the patient 90 which may be estimated from the Bio-Z signals detected by the sensors 106 of patches 102. The reconstruction of the remaining physiological parameter signals 225 (excluding the physiological parameter signal 225 corresponding to the respiratory signal of the patient 90) by removing the contribution of the RR extracted by the SOBI algorithm for each demodulated signal 215 using a demixing matrix which is the inverse of the determined mixing matrix A. The resulting, demixed demodulated signals 215 include temporal information of the heart activity (e.g., HR, etc.) of the patient 90 without any disturbance of the phase characteristics (i.e. delayed arrival of heart pressure pulse wave at different locations of the thorax due to finite pulse wave velocity). For example, and not intending to be bound by any particular theory, a demixing matrix D may calculated in accordance with Equations (6) and (7) presented below where I corresponds to an identify matrix of the D and A matrices:

I=D×A (6)

v(t)=D×y(t) (7)

The SOBI algorithm may employ the demixed demodulation signals 215 to reconstruct or estimate one or more physiological parameter signals 225 corresponding to the heart activity of the patient 90, from which a HR of the patient may be derived. For example, and not intending to be bound by any particular theory, a physiological parameter signal 225 corresponding to the heart activity (BioZH(t)) of patient 90 may be extracted in accordance with Equation (8) below where the contribution of the respiratory activity (BioZR(t)) is subtracted:

BioZH(t)=v₁(t)−Dₙₓ₁×y(t) (8)

A spectrogram of each physiological parameter signal 225 corresponding to the heart activity of the patient 90 (e.g., BioZH(t)) may be extracted using a fast Fourier transform (FFT) and a dominant frequency region may thereby be detected. A LPF (e.g., a second-order Butterworth LPF) may then be applied with a cut-off frequency that is approximately 1 Hz higher than the detected dominant frequency to reject high-frequency oscillations. To calculate the HR, zero-crossing, foot and peak points of the first and second derivatives of the signal may be used to detect the important characteristic points, such as local peaks, and feet and maximum slope points (MSPs). These points may be used to calculate the corresponding interbeat intervals (IBI) of the patient 90, where IBI may comprise the period or the duration of one heartbeat of the patient 90. In order to reduce the effect of motion artifacts, a moving average filter may be applied to the inverse of the calculated IBIs (1/IBI, beats per second) with an approximately 30-second averaging window and approximately 28 seconds of overlap to the physiological parameter signals 225. The output of each window may be multiplied by approximately 60 to obtain the HR of the patient 90 in bpm for each 30-second window.

In order to evaluate the performance of the system (e.g., system 100 shown in FIG. 6), wirelessly communicable, Bio-Z detecting patches (e.g., patches sharing features in common with the patches 102 shown in FIGS. 7, 8) were used to extract continuous HR and RR values from ten healthy human subjects. Prior to the main experimental study, the performance improvement with the body GND electrode placement was first evaluated and two pilot studies were conducted to decide on the optimum patch size and to show the importance of multiple patch approach rather than single patch measurements. As discussed further below, a smaller electrode separation on a single patch may lead to a direct decrease in the size of the patch supporting the wearable applications built on top of this technology with a trade-off in the SNR. In addition, the multiple patch approach proposed herein may provide high-fidelity separation of heart and lung activities.

As described above, a novel sensing technique was implemented with Bio-Z detecting patches to provide global observations of the chest physiology. We achieved through two general steps: First, a patch (“TX patch”) comprising a current transmitter was placed underneath the left armpit. Second, with the assumption that all parts of the upper body see a fraction of this current, three patches (the “RX patches”) including voltage sensors were placed at different locations of the upper chest. A first patch (“Bio-Z1”) comprising voltage sensors was placed across the heart (left of the sternum), the second patch (“Bio-Z2”) comprising voltage sensors was placed at the right of the sternum, and a third patch (“Bio-Z3”) comprising voltage sensors was placed underneath the right armpit, giving full coverage across the upper chest. In all of the Bio-Z measurements conducted during these experiments, a sinusoidal AC signal at 10 kHz with 0.64 mA RMS amplitude was injected, complying with the safety standards.

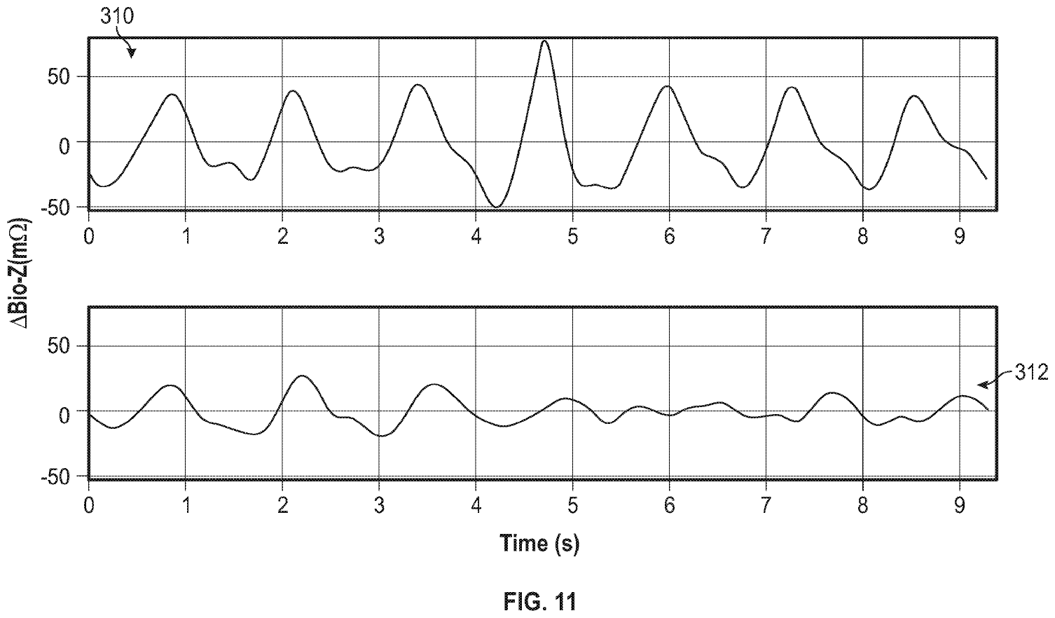

To evaluate the effect of GND electrode placement of the RX patches on the body, two one-minute long experiments were run with the only difference between the experiments being the inclusion of the GND electrode. For both experiments, the TX patch was used for the injection and Bio-Z1 for sensing. Since the voltage pick-up is differential, the location of the GND electrode may not be significant in at least some applications. On the other hand, the GND electrode was placed next to the RX patch to prevent the patch size increase. Under the same injection amplitudes, the SNR of the carrier signal, picked-up by the TX patch with the body GND electrode, was observed to be 7 dB higher than the case without the body GND electrode given that the GND electrode may improve the common-mode rejection of the IA. Referring to FIG. 11, graphs 310, 312 illustrating a flipped ΔBio-Z signal after demodulation are shown for both cases. The signal fidelity may be better with the body GND electrode (with body GND for graph 310 and without body GND for graph 312), in agreement to the SNR measurements. Consequently, moving forward the body GND electrodes placed next to the RX patches were used with no constraint on the precise placement.

For the pilot study regarding the size of the Bio-Z detecting patches, the effect of the separation amount between differential electrodes (nodes) of each patch was tested from 1-inch to 5-inches under a total of 25 minutes of data collection from a single subject. Moving forward, 3-inches separation was used between the two differential nodes of each TX and RX patches, due to its superior performance (in some applications) compared to the smaller sizes while providing similar performance compared to the larger sizes. In order to complete an extensive evaluation of the Bio-Z detecting patches, four sets of five-minute continuous data were collected from 10 healthy subjects.

To assess the performance of Bio-Z detecting patches for estimating the HR, an ECG signal captured simultaneously by the Bio-Z1 sensor placed at the heart was used to detect the true heartbeats. Moreover, capnography (e.g., RespSense II, Nonin, USA) was used for measuring a reference of the respiration waves recorded during the data collection with Bio-Z detecting patches to assess the estimation of RR. The capnography tracked the CO₂ concentration through a nasal cannula connected to the device to determine the RR. During the data collection process, subjects remained seated with the capnography device connected through a nasal cannula. Prior to the start of the actual data collection with the Bio-Z detecting patches, we asked subjects were asked to hold their breath for 10 seconds, where we used this signature time interval to synchronize capnography data to the Bio-Z detecting patches data.

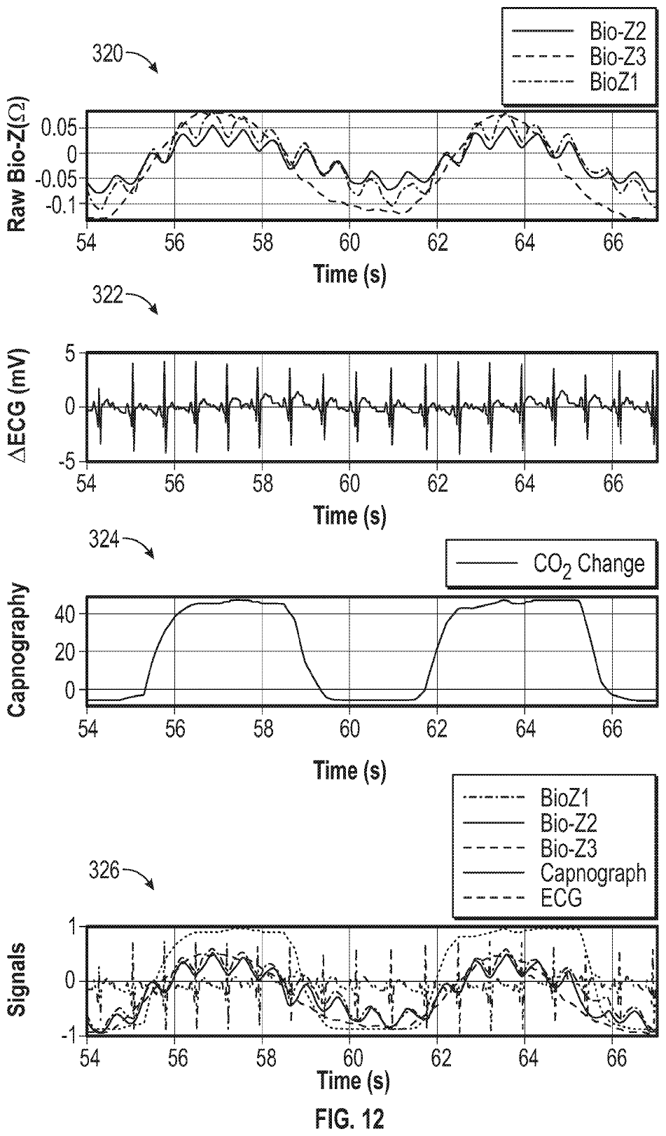

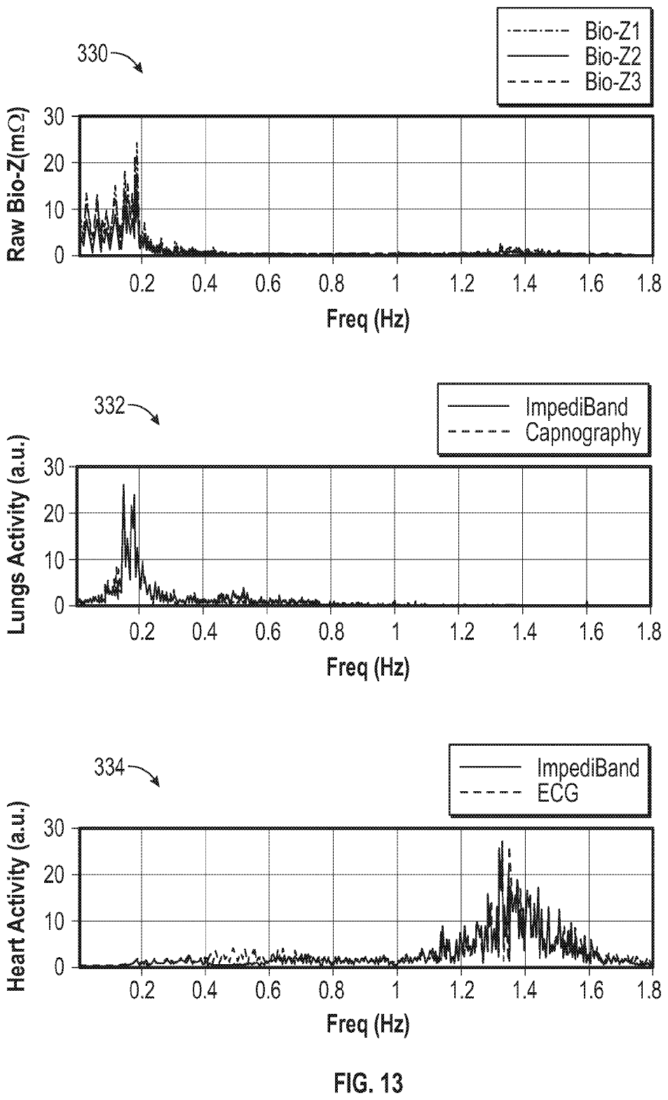

Referring to FIG. 12, graphs 320, 322, 324, and 326 are shown which illustrate exemplary physiological parameters or signals measured with the experimental setup and the capnography device are shown. Particularly, graphs 320, 322 indicate time plots of measured physiological signals with the Bio-Z detecting patches, graph 324 indicates a reference capnography measurement, and graph 326 indicates all signals scaled and plotted within the same time legend. Periodic lung movements due to respiration were observed on the raw Bio-Z signals captured by the Bio-Z detecting patches. Based on these observations, it is expected that iterative SOBI would identify this temporal correlation between the sensors with high fidelity. Referring to FIG. 13, graphs 330, 332, and 334 are shown which illustrate an exemplary frequency spectrogram correlating the raw Bio-Z inputs which are inputted to iterative SOBI and the respiration and heart activity signals obtained through iterative SOBI. Particularly, graph 330 indicates raw Bio-Z signals modulated by the respiration cycle with additional frequency components appearing at higher frequencies; graph 332 indicates a respiration signal obtained after the application of source separation which matches a reference signal acquired through capnography; and graph 334 indicates a hear activity signal estimated after signal reconstruction which exhibits an improved SNR matching with a reference ECG signal. Corresponding references were also plotted for both sources in FIG. 13. After the application of the iterative SOBI algorithm with the signal reconstruction, the system estimated heart and lung activities with high accuracy. The separated heart (Bio-ZH) and respiration (Bio-ZR) source signals (y(t)) were used in HR and RR estimations, respectively.

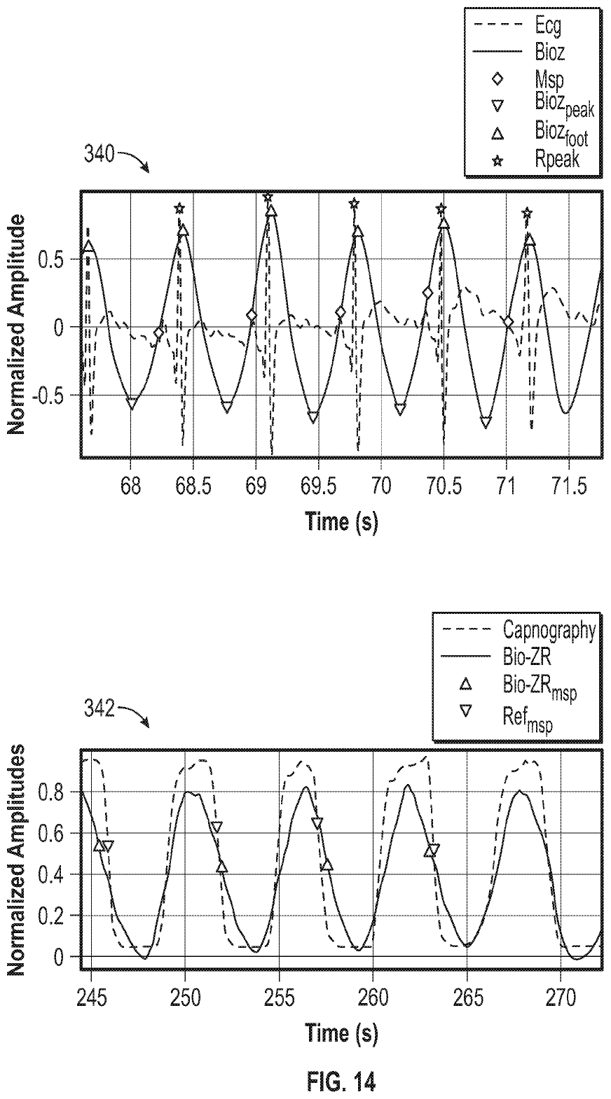

Referring to FIG. 14, graphs 340, 342 are shown which illustrate an example of the extracted characteristics points on the Bio-ZH and Bio-ZR signals and the corresponding reference signals are shown. Particularly, graph 340 indicates Bio-ZH with reference to ECG while graph 342 indicates Bio-ZR with reference to capnography signals. IBIs were calculated from a combination of maximum slope point (MSP), peak and footpoints in Bio-ZH and R-peaks in the ECG signal. To estimate IBrIs from the Bio-ZR and capnography signals, MSPs were used. In order to mitigate the effect of motion artifacts and high-frequency oscillations that alter the peak points, a moving average algorithm, similar to the moving average algorithms described above, were applied.

TABLE I

| | | | IBrI | | |
|---|---|---|---|---|---|
| Patch | RR | | Upper | Lower | |
| Size | | RMSE | 95% | 95% | |
| (inches) | r | (BPM) | limit (s) | limit (s) | r |
| 5 | 0.961 | 0.167 | 0.444 | −0.389 | 0.961 |
| 4 | 0.914 | 0.112 | 0.439 | −6.464 | 0.914 |
| 3 | 0.912 | 0.067 | 0.274 | −0.283 | 0.912 |
| 2 | 0.904 | 0.299 | 0.785 | −0.605 | 0.904 |
| 1 | 6.962 | 0.183 | 0.549 | −0.482 | 0.962 |

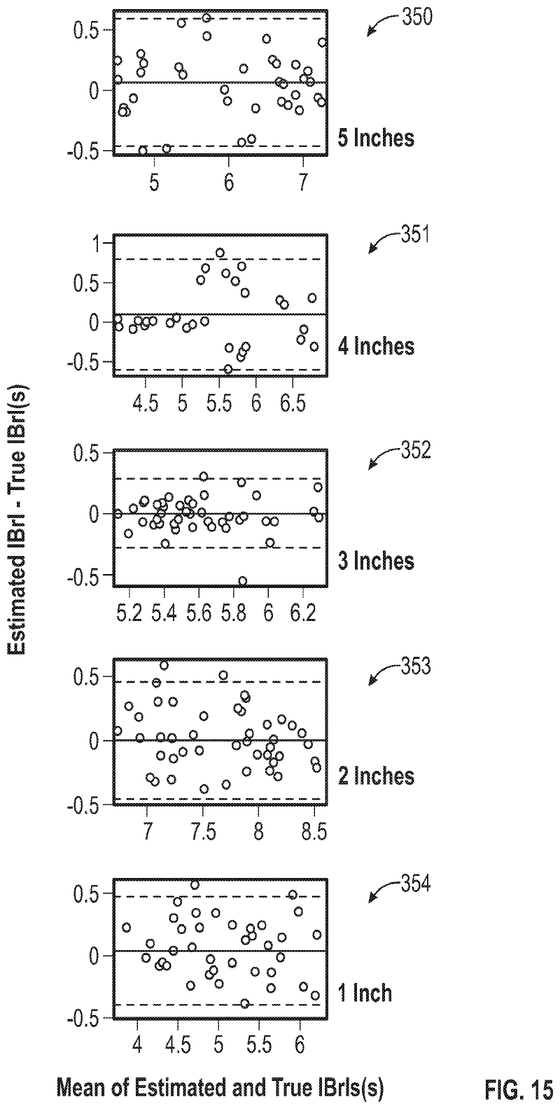

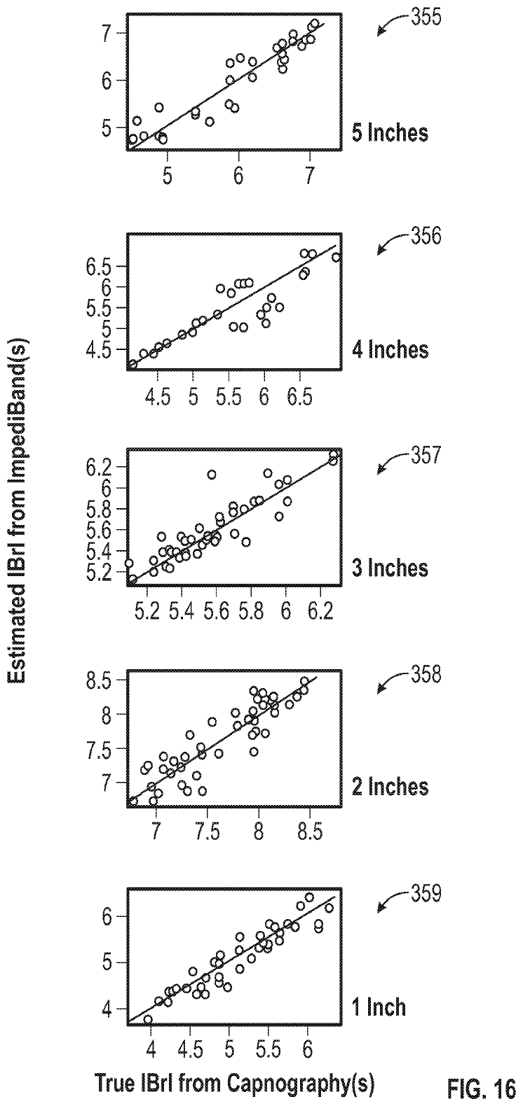

The Pearson's correlation coefficient (r) and average root mean square error (RMSE) in breaths-per-minute (BPM) in RR estimation for five different patch sizes determined by the separation between the differential electrodes of each patch starting from 5-inches up to 1-inch are shown above in Table I. Referring to FIGS. 15, 16, graphs 350-359 are shown which illustrate the Bland-Altman and Pearson's correlation analyses for this experiment. Particularly, graphs 350-354 illustrate the Bland-Altman correlation analysis plots while graphs 355-359 illustrate the Pearson's correlation analysis plots. Graphs 350, 355 pertain to a patch size of approximately 5 in; graphs 351, 356 pertain to a patch size of approximately 4 in; graphs 352, 357 pertain to a patch size of approximately 3 in; graphs 353, 358 pertain to a patch size of approximately 2 in; and graphs 354, 359 pertain to a patch size of approximately 1 in. For each plot, 51 IBrIs calculated from the averaged peak-to-peak values were used. It was observed from the experiment that the Bio-Z detecting patches provide strong confidence in RR estimation with all patch sizes with a minimum of 0.9 correlation and a maximum of 0.3 BPM RMSE.

TABLE II

| | | | IBI | | |
|---|---|---|---|---|---|
| | | | Upper | Lower | |
| Patch | HR | | 95% | 95% | |
| Size | | RMSE | limit | limit | |
| (inches) | r | (bpm) | (ms) | (ms) | r |
| 5 | 0.976 | 0.267 | 6.16 | −6.95 | 0.976 |
| 4 | 0.980 | 0.325 | 9.15 | −4.87 | 0.984 |
| 3 | 0.983 | 0.655 | 17.1 | −18.9 | 0.983 |
| 2 | 0.751 | 3.597 | 124.3 | −25.6 | 0.765 |
| 1 | −0.122 | 5.217 | 189.2 | −67.2 | −0.090 |

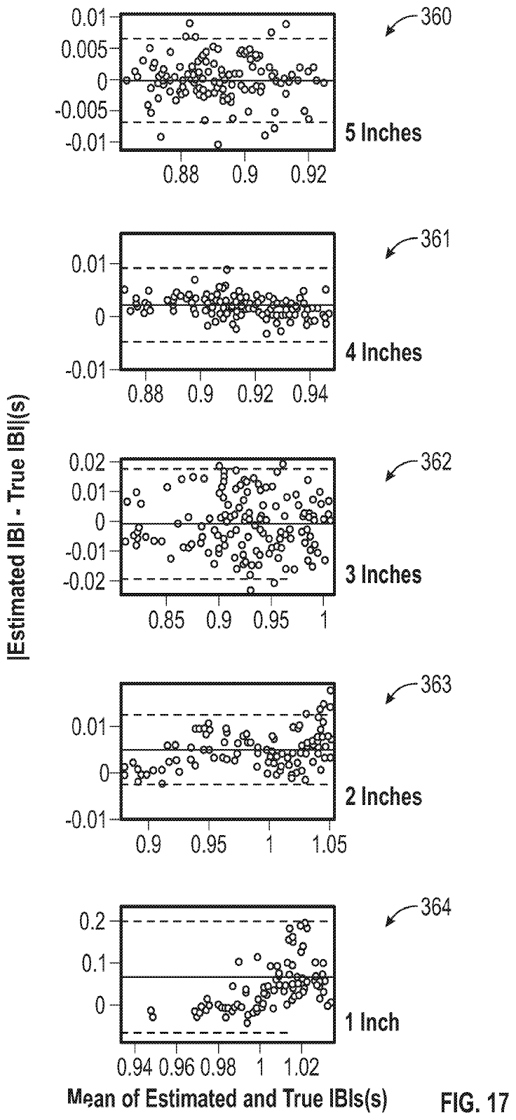

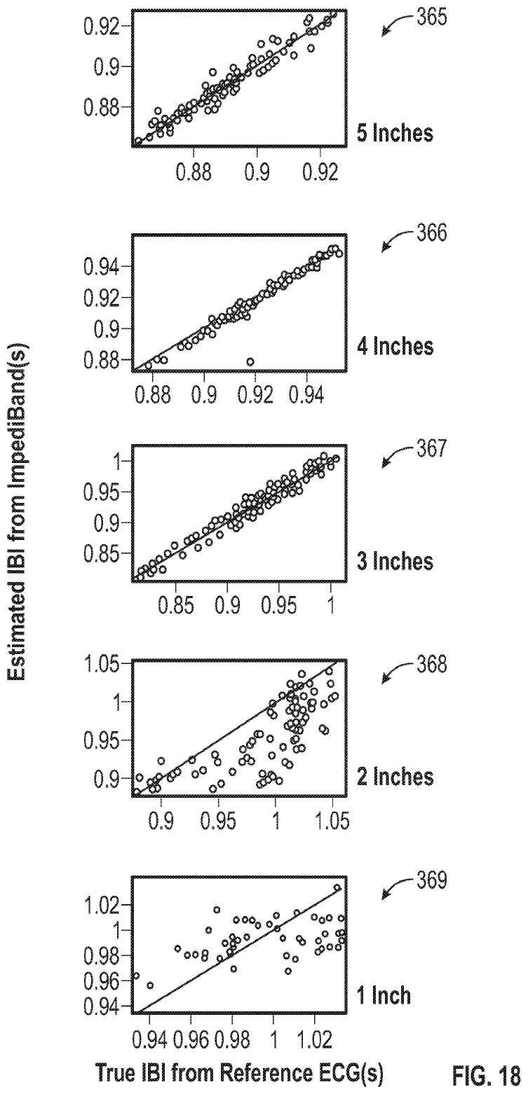

The r and RMSE in bpm in HR estimation for all patch sizes are shown above in Table II. Results show that the device performance with higher than 3-inches patch separation remained comparable with an RMSE of less than one bpm. However, in contrast to RR estimation performance, further decreasing the size from 3-inches to 2-inches degraded the signal quality and increases the error in HR estimation. Referring to FIG. 17, 18, graphs 360-369 are shown which illustrate the corresponding Bland-Altman and Pearson's correlation plots for different patch sizes using 140 IBIs for each patch size. Particularly, graphs 360-364 illustrate the Bland-Altman correlation analysis plots while graphs 365-369 illustrate the Pearson's correlation analysis plots. Graphs 360, 365 pertain to a patch size of approximately 5 in; graphs 361, 366 pertain to a patch size of approximately 4 in; graphs 362, 367 pertain to a patch size of approximately 3 in; graphs 363, 368 pertain to a patch size of approximately 2 in; and graphs 364, 369 pertain to a patch size of approximately 1 in. The 95% limits of agreement are also shown above in Table II. A 3-inch patch size was used moving forward with the extensive analysis due to the high accuracy in both HR and RR estimations (in some applications) using this configuration.

1377 IBrIs were used in the analysis, where data for subjects 7 and 9 in the analysis were excluded due to peak detection problems and very high noise in the dataset for these subjects, respectively.

TABLE III

| | | RMSE |
|---|---|---|
| Subject | r | (BPM) |
| 1 | 0.978 | 0.097 |
| 2 | 0.893 | 0.294 |
| 3 | 0.919 | 0.292 |
| 4 | 0.892 | 0.364 |
| 5 | 0.935 | 0.128 |
| 6 | 0.962 | 0.559 |
| 8 | 0.848 | 0.365 |
| 10 | 0.944 | 0.181 |
| Average | 0.921 ± 0.042 | 0.285 ± 0.151 |

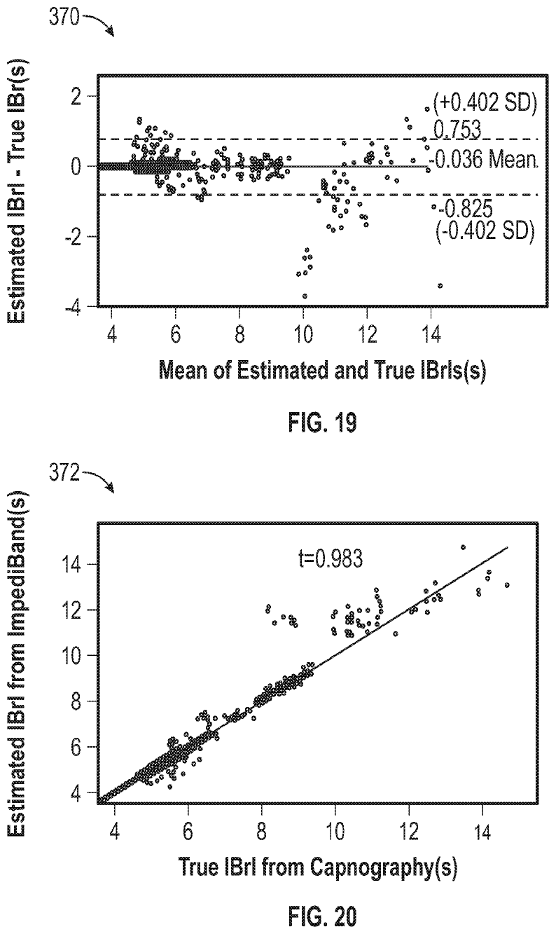

r, RMSE in BPM and average BPM are presented above for each subject in Table III. Referring to FIGS. 19, 20, graphs 370, 372 are shown which illustrate plots for Bland-Altman correlation analysis (graph 370 of FIG. 20) and Pearson's correlation analysis (graph 372 of FIG. 21) over 1377 IBrIs. Graphs 370, 372 indicate agreement between the novel method described herein and the reference method, where the negative and positive 95% limits of agreement values appeared less than 1.3 BPM, with the mean of the error (IBI_{estimated}−IBIₜᵣᵤₑ) appearing as 0.07 BPM. In addition, Pearson's correlation analysis resulted in 0.983 for the correlation coefficient, r.

In the performance evaluations, 3798 IBIs calculated using more than 2 hours of data collection in total from seven healthy subjects were used. For this evaluation, data corresponding to Subjects 1 and 9 were excluded due to high divergence from the rest of the subjects. In addition, an anomaly in ECG of Subject 5 was observed, also detected with the Bio-Z detecting patches. For this reason, Subject 5's data was separated from the dataset. To indicate the agreement between the ECG and Bio-ZH acquired with the Bio-Z detecting patches, the averaged IBIs for both signals were compared.

TABLE IV

| | | RMSE |
|---|---|---|
| Subject | r | (BPM) |
| 1 | 0.978 | 0.097 |
| 2 | 0.893 | 0.294 |
| 3 | 0.919 | 0.292 |
| 4 | 0.892 | 0.364 |
| 5 | 0.935 | 0.128 |
| 6 | 0.962 | 0.559 |
| 8 | 0.848 | 0.365 |
| 10 | 0.944 | 0.181 |
| Average | 0.921 ± 0.042 | 0.285 ± 0.151 |

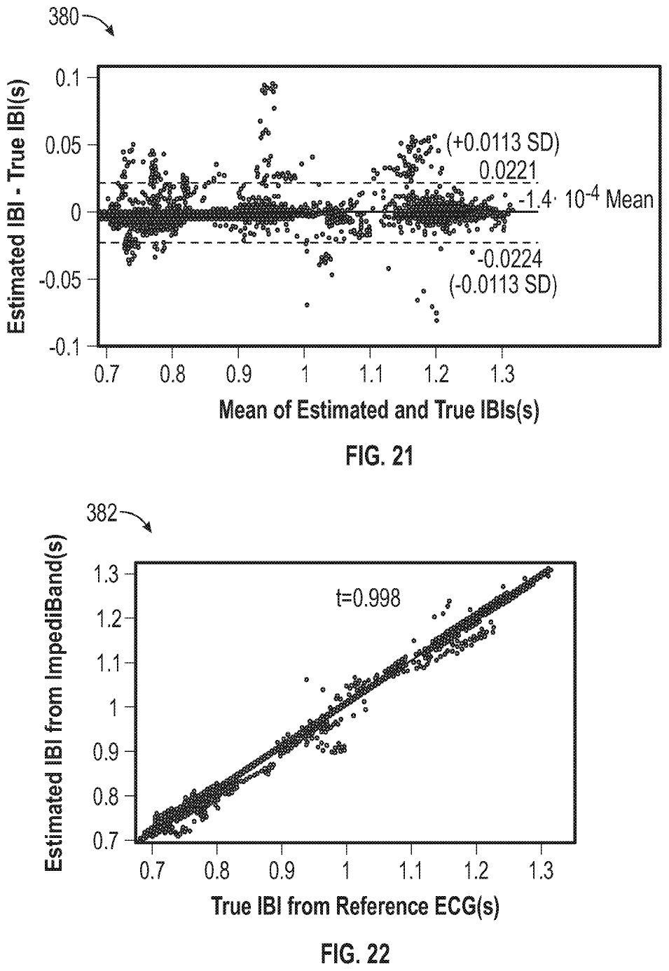

Table IV shown above provides the average RMSE in bpm and r for each subject in HR estimation. The results demonstrated a strong correlation of estimated HR with the reference HR for each subject, with average r appearing at 0.948 and RMSE at 0.579 bpm. In order to conduct a comprehensive analysis of the IBI estimation performance, the Bland-Altman and Pearson's correlation analyses were carried over 3798 IBIs. Referring to FIGS. 21, 22, graphs 380, 382 are shown which illustrate the Bland-Altman correlation plot (graph 380) and the Pearson's correlation plot (graph 382). Graphs 380, 382 indicate a strong agreement between the novel method described herein and the gold standard. The 95% limits of the agreement appear at 22.1 ms and −22.4 ms in graph 380. The Pearson's correlation plot shown in graph 382 includes a Pearson's correlation coefficient of 0.998.

In order to evaluate the performance improvement with the novel method disclosed herein with respect to the traditional methods that depend on a single patch measurement, a pilot study was conducted on a single subject over 20 minutes of data collection. With the subjects at rest, Bio-Z detecting patches were placed in a configuration similar to the experiment described above and a lock-in based demodulation was carried out separately for each voltage sensor to extract the raw Bio-Z signals. Three different methods were then defined to extract the heart activity. The first method comprised running a 2nd order Butterworth filtering with a cut-off at 0.5 Hz on a single patch Bio-Z, whereas second and third methods depended on the application of iterative SOBI on two patches and three patches respectively. To evaluate the performance in separating the heart activity, precision and recall analysis were performed on estimated peak locations from the output signal and the true peak locations extracted from the ECG, as well as the Bland-Altman and Pearson's correlation analyses on the estimated IBIs and the true IBIs. For the filtering case with a single patch, the patch placed on the heart was chosen to get the strongest heart activity, and for two patches based iterative SOBI, we tried all three input combinations and took the average.

1988 beats were used in the analysis, where the peak detection algorithm described herein was applied to the output signal for each method and the reference ECG signal. Based on the time location of each of the estimated peak with respect to the reference peak, the peaks were classified as true positive (TP), false positive (FP) or false negative (FN). The TP, FP, and FN refer to the cases where the estimation falls in between 20% of the current beat in reference to ECG peak, falls outside of this 20% threshold and no peak is found inside this threshold, respectively. The accuracy and precision were then calculated according to Equations (9) and (10) presented below:

$$\begin{matrix}Accuracy = \frac{TP}{TP + FP} & ( 9 ) \\ Precision = \frac{TP}{TP + FN} & ( 10 )\end{matrix}$$

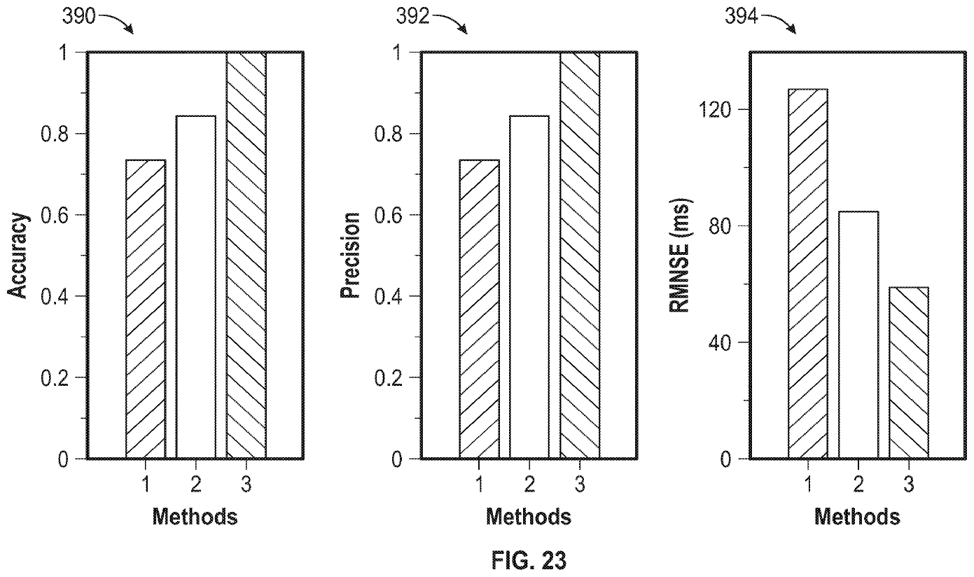

After this classification, the RMSE based on the TPs only was also calculated, where the error is defined as the difference between the IBIs calculated through reference versus estimated peaks. Referring to FIG. 23, charts 390, 392, and 394 are shown which illustrate the accuracy (shown in chart 390), precision (shown in chart 392), and RMSE (shown in chart 394) obtained for each method are shown for a single patch configuration, a two patch configuration, and a three patch configuration (the number of patches arranged on the X-axis), where SOBI performed with three patches performed the best in all analysis metrics. In addition, SOBI even with two patches showed a significant improvement compared to the traditional filtering approach.

In this analysis, instead of classifying estimated peaks with respect to the reference peak, for each reference peak, the closest estimated peak was chosen, followed by beat-by-beat IBI calculation for both reference and estimated signals. 1295 IBIs were used in the analysis.

TABLE V

| | IBI | | | | |
|---|---|---|---|---|---|
| | Lower | | Upper | | |
| | 95% | Mean | 95% | | |
| | limit | Error | limit | | RMSE |
| Method | (ms) | (ms) | (ms) | r | (%) |
| 1 Patch | −107.12 | 3.95 | 115.03 | 0.8658 | 6.1% |
| with 2^{nd} | | | | | |
| order | | | | | |
| Butterworth | | | | | |
| HPF | | | | | |
| 2 Patches | −108.85 | 1.14 | 111.13 | 0.8780 | 6.0% |
| with | | | | | |
| iterative | | | | | |
| SOBI | | | | | |
| 2 Patches | −81.87 | 1.68 | 85.24 | 0.9184 | 4.5% |
| with | | | | | |
| iterative | | | | | |
| SOBI | | | | | |

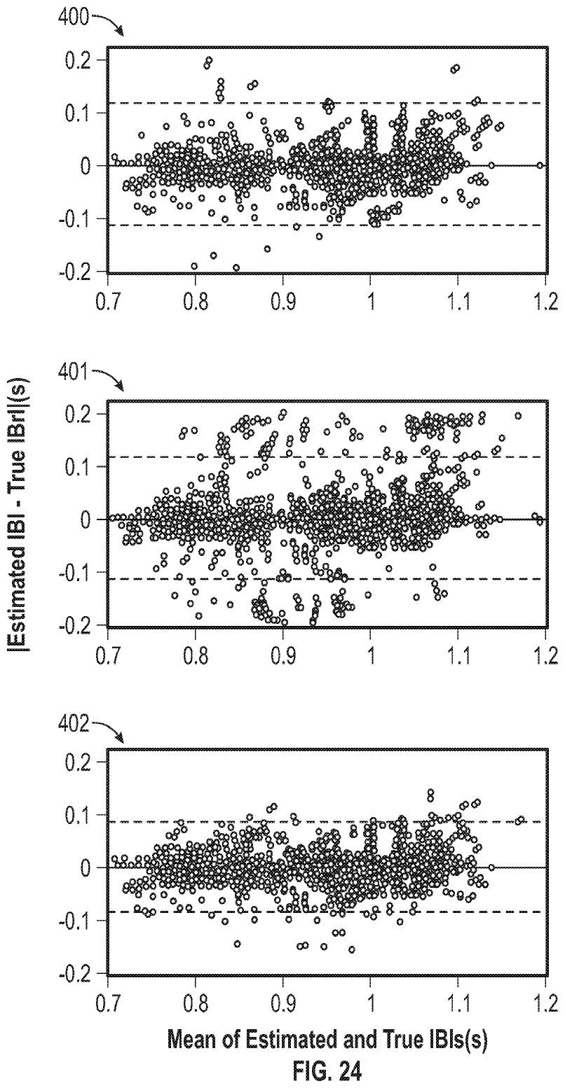

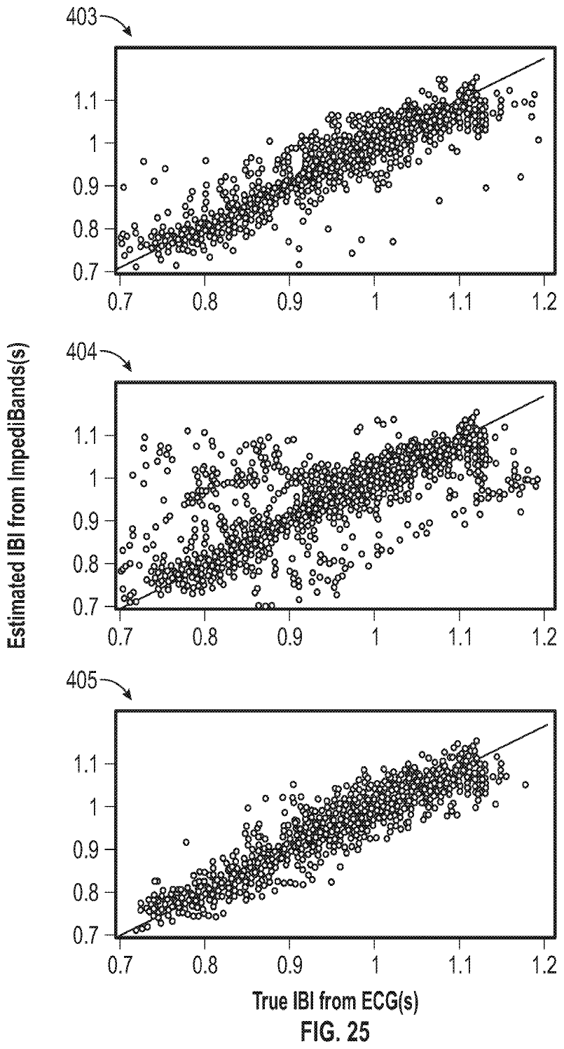

r, RMSE and Bland-Altman coefficients are shown above in Table V. Referring to FIGS. 24. 25, graphs 400-406 are shown which illustrate the plots for the Bland-Altman correlation analysis (graphs 400-402) and the Pearson's correlation analysis (graphs 403-405) over all IBIs are shown. Graphs 400, 403 pertain to a single patch system; graphs 401, 404 pertain to a system including two patches; and graphs 402, 405 pertain to a system including three patches. The results show a significant improvement in the agreement between the novel method described herein and the reference method compared to filtering.

Overall the results of the above described experimentation demonstrate that Bio-Z detecting patches perform HR and RR estimations high accurately with an average RMSE of 0.288 BPM and 0.589 bpm for HR and RR, respectively. The above results also demonstrate the performance of the system under various electrode separation configurations and patch sizes. The above described pilot study indicated that the 3-inches patch separation is sufficient for high fidelity signal acquisition. Moreover, it is still possible to use 1-inch patches to extract the respiration rate.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A system for monitoring one or more physiological parameters, comprising:
a plurality of patches positionable at a plurality of locations on a surface of a body, wherein the plurality of patches are electrically isolated from each other;
wherein a first patch of the plurality of patches comprises a transmitter configured to inject an electrical signal into the body at a first location on the surface of the body; and
wherein a second patch of the plurality of patches comprises a sensor configured to detect a bio-impedance signal at a second location on the surface of the body that is spaced from the first location, and wherein the bio-impedance signal is induced within the body by the electrical signal injected from the first patch which is electrically isolated from the second patch.

2. The system of claim 1, wherein:
the transmitter of the first patch is configured to inject an alternating current at a first frequency;
the sensor of the second patch is configured to detect the bio-impedance signal from a voltage induced by the injected alternating current; and
the second patch comprises a transmitter configured to inject an alternating current at a second frequency that is different from the first frequency.

3. The system of claim 2, wherein the first patch comprises sensor configured to detect a bio-impedance signal from the body that is induced by the alternating currents injected from the first patch and the second patch.

4. The system of claim 1, further comprising a controller coupled to the second patch and configured to estimate the one or more physiological parameters based on the bio-impedance signal.

5. The system of claim 4, wherein the controller is configured to adjust at least one of a frequency, an amplitude, and a shape of the electrical signal injected by the transmitter.

6. The system of claim 4, wherein at least one of the one or more physiological parameters estimated by the controller corresponds to at least one of heart activity and respiratory activity of a patient.

7. The system of claim 4, wherein the one or more physiological parameters comprises a plurality of physiological parameters, and wherein the controller is configured to apply a source separation algorithm on the bio-impedance signal to separate a first physiological parameter from the plurality of physiological parameters.

8. The system of claim 7, wherein the controller is configured to apply a demodulation algorithm to the bio-impedance signal prior to the application of the source separation algorithm to the bio-impedance signal.

9. The system of claim 1, wherein each of the plurality of patches are independently electrically grounded to the body.

10. The system of claim 9, wherein each of the plurality of patches comprises a ground electrode in electrical contact with the surface of the body.

11. The system of claim 1, wherein at least one of the plurality of patches comprises a sensors configured to independently detect a bio-impedance signal from the body induced by the electrical signal injected from the second patch.

12. The system of claim 1, wherein:
the transmitter of the first patch is configured to inject an alternating voltage at a first frequency;
the sensor of the second patch is configured to detect the bio-impedance signal from a current induced by the injected alternating voltage;
the second patch comprises a transmitter configured to inject an alternating voltage at a second frequency that is different from the first frequency; and
the first patch comprises sensor configured to detect a bio-impedance signal from the body that is induced by the alternating voltages injected from the first patch and the second patch.

13. A system for monitoring one or more physiological parameters, comprising:
a plurality of patches configured to releasably couple with a surface of a body at a plurality of locations, wherein each of the plurality of patches are independently electrically grounded to the body;
wherein a first patch of the plurality of patches comprises a transmitter configured to inject an electrical signal into the body at a first location on the surface of the body; and
wherein a second patch of the plurality of patches comprises a sensor configured to detect a bio-impedance signal at a second location on the surface of the body that is spaced from the first location, and wherein the bio-impedance signal is induced within the body by the electrical signal injected from the first patch.

14. The system of claim 13, wherein each of the plurality of patches comprises a ground electrode in electrical contact with the surface of the body.

15. The system of claim 13, wherein:
the transmitter of the first patch is configured to inject an alternating current at a first frequency;
the sensor of the second patch is configured to detect the bio-impedance signal from a voltage induced by the injected alternating current;
the second patch comprises a transmitter configured to inject an alternating current at a second frequency that is different from the first frequency; and
the first patch comprises sensor configured to detect a bio-impedance signal from the body that is induced by the alternating currents injected from the first patch and the second patch.

16. The system of claim 13, further comprising:
a controller coupled to the second patch and configured to estimate the one or more physiological parameters based on the bio-impedance signal;
wherein the one or more physiological parameters comprises a plurality of physiological parameters, and wherein the controller is configured to apply a source separation algorithm on the bio-impedance signal to separate a first physiological parameter from the plurality of physiological parameters.

17. The system of claim 16, wherein the controller is configured to adjust at least one of a frequency, an amplitude, and a shape of the electrical signal injected by the transmitter.

18. A method for monitoring one or more physiological parameters, comprising:
(a) injecting an electrical signal into a body with a first patch positioned at a first location on a surface of the body; and
(b) detecting a bio-impedance signal induced within the body by the injected electrical signal with a second patch positioned at a second location on the surface of the body and that is electrically isolated from the first patch, wherein the second location is spaced from the first location.

19. The method of claim 18, further comprising:
(c) independently electrically grounding the first patch and the second patch to the surface of the body.

20. The method of claim 18, further comprising:
(c) estimating the one or more physiological parameters based on the detected bio-impedance signal.
